(19) United States
(12) Patent Application Publication
(10) Pub. No.: US 2020/0135336 A1
(43) Pub. Date: Apr. 30, 2020

(54) METHOD AND APPARATUS FOR DETERMINING AND PRESENTING INFORMATION REGARDING MEDICAL CONDITION LIKELIHOOD

(71) Applicant: Medbaye LLC, Maynard, MA (US)

(72) Inventors: Eric Howard Gluck, Chicago, IL (US); Matthew Thomas Tyndall, Boxborough, MA (US); Robert Kevin Moore, Natick, MA (US)

(73) Assignee: Medbaye LLC, Maynard, MA (US)

(21) Appl. No.: 16/661,263

(22) Filed: Oct. 23, 2019

Related U.S. Application Data
(60) Provisional application No. 62/912,269, filed on Oct. 8, 2019.
(60) Provisional application No. 62/830,681, filed on Apr. 8, 2019.
(60) Provisional application No. 62/749,762, filed on Oct. 24, 2018.

(51) Int. Cl. G16H 50/20; G16H 10/20; G16H 15/00

(52) U.S. Cl. CPC G16H 50/20; G16H 15/00; G16H 10/20

(57) ABSTRACT

A method and system for determining a composite post-test likelihood that a patient has a medical condition using an iterative Bayesian analysis of test information and test results for multiple tests. Multiple medical conditions can be assessed and results viewed simultaneously, including assessments performed based on hypothetical tests and test results. Such assessment may help guide a clinician to selecting tests and/or treatment that are particularly relevant to a patient's medical condition diagnosis.

### RELATED APPLICATIONS

This Application claims priority under 35 U.S.C. § 119(e) to U.S. Provisional Application Nos. 62/749,762 filed Oct. 24, 2018, 62/830,681 filed Apr. 8, 2019, and 62/912,269 filed Oct. 8, 2019, the contents of each of which is herein incorporated by reference in its entirety.

### BACKGROUND

Bayesian statistics provides an effective method for quantifying uncertainty. This makes Bayesian statistics helpful for analyzing the uncertainty of diagnostic test results. Discrete tests that provide a “positive” or “negative” test result (such as for pregnancy), will typically have a stated accuracy of positive and negative results based on clinical studies. Non-discrete or continuous tests (such as body temperature), may have a threshold value to convert a continuous result into a discrete value (e.g., “consider a patient with a temperature of 100° F. or higher as positive for a fever.”). For example, a test may have a stated accuracy of posterior results of 90%, indicating that out of 100 tests given to patients known to be positive, 90 will receive a positive result and 10 a negative result. Many clinicians assume this means there is a 90% chance that a patient with a positive test result is, in fact, positive. But a more sophisticated analysis shows this to be incorrect.

Using Bayesian statistics to analyze test results allows us to address this cognitive bias so the clinician can arrive at a more realistic probability. The stated accuracy of a test addresses the question “given that the patient is known positive, what is the probability the test will give a positive result?”; Bayesian statistics allows the reverse of this by providing an answer to the question, “given that the test result is positive, what is the probability that the patient actually has the disease?”

To arrive at the answer, we must take into account not only the true positive accuracy of the test, but also any false positives among the true negative patients. The ratio of these two, weighted by the relative size of each group, provides the answer to the question posed above.

To understand the clinical significance of this analysis, consider an example. Imagine a randomly selected population of 1,000 patients of whom 100 are truly positive and 900 are truly negative. If our test has a 90% true positive rate (also known as sensitivity) and an 85% true negative rate (also known as specificity), then 90 of the true positive patients (90% of 100) and 135 of the true negative patients (15% of the 900) will have positive test results, for a total of 225 “positives.” Therefore, Bayesian statistics reveal that a patient with a positive result for this test has only a 40% probability [90/(90+135)] of having the disease we are testing for—not the 90% probability that many clinicians might assume given the test's stated accuracy.

This gap in statistical knowledge, common among most clinicians whose training does not focus on biostatistics can lead to incorrect assessments of test results and, potentially, to diagnostic error and inappropriate treatment decisions. Appropriate application of Bayesian statistics, in a form that clinicians can easily understand, can help dramatically improve their understanding of the true likelihood that a patient has a suspected medical condition based on available test results, thus improving diagnostic accuracy.

### SUMMARY OF INVENTION

One embodiment of the invention relates to a method and/or system for assisting medical clinicians or other users in determining the likelihood that a patient has a suspected medical condition based on a statistical assessment of the results of one or more diagnostic laboratory or other tests. The likelihood that the patient has the medical condition may be determined using an iterative Bayesian analysis of results from multiple tests where the post-test likelihood determined by analysis of a first test is used as the pre-test likelihood when determining a post-test likelihood for a second test, and so on. This iterative Bayesian analysis provides a composite likelihood that the patient has the medical condition based on all tests and test results, irrespective of the order in which test results are analyzed or the number of tests analyzed. This can provide a user with a powerful ability to assess whether a patient has a particular medical condition based on a confusingly large set of tests and/or test results that may be conflicting. By helping clinicians more accurately diagnose patients, treatments can be more efficiently selected and implemented faster, avoiding wasteful or needless procedures.

In some embodiments, one or more tests may be actually performed with respect to the patient, e.g., blood drawn from the patient and tested for a particular biomarker, and one or more tests may be hypothetical, i.e., not actually performed with respect to the patient but rather have test result values picked by the user or otherwise determined. This may allow a clinician or other user to assess the value of performing the hypothetical test, e.g., by evaluating the determined likelihood that the patient has a medical condition for different results for the hypothetical test. Where a hypothetical test is shown by iterative Bayesian analysis to have little effect on the composite post-test likelihood that a particular medical condition is present, a user may decide the test is not worthwhile, and vice versa. This can allow users to avoid unnecessary or useless tests, expensive tests that do not yield more information than less costly tests, as well as confirm in advance whether a test will provide useful results in determining whether a particular medical condition is present. While in some embodiments a clinician may select a hypothetical test and direct analysis of selected results for the hypothetical test, a computerized system may be arranged to automatically assess one or more hypothetical tests using selected results for the hypothetical tests to make a recommendation that one or more tests be performed with respect to a patient. For example, test results for a patient may be entered into the system and a composite post-test likelihood that the patient has one or more medical conditions may be determined and displayed for assessment by a clinician. In addition, the computerized system may determine composite post-test likelihoods for the medical condition(s) based on one or more hypothetical tests and selected test results for the hypothetical tests. The composite post-test likelihoods for the hypothetical tests may be analyzed, and hypothetical tests identified which may provide conclusive or otherwise useful results regarding a likelihood that the patient has a particular medical condition. As an example, the computerized system may determine post-test likelihoods for multiple hypothetical tests and identify hypothetical tests and test results which return a composite post-test likelihood over a threshold (e.g., 90% to rule-in the condition) and/or under a threshold (e.g., under 10% to rule-out a condition) may be displayed to a user as a recommended test to be performed. This technique can help clinicians determine which tests should be performed and which tests will provide the most useful information for diagnosing one or more medical conditions.

In other embodiments, the likelihood that a patient has two or more medical conditions may be assessed using an iterative Bayesian analysis, e.g., based on a same set of tests. The likelihood information for the two or more medical conditions determined by analysis of the test results may be displayed to a user, e.g., so the user can view side-by-side the likelihood that the patient has a first medical condition and/or a second medical condition (or more medical conditions). This can help the user determine which medical condition is more likely to be present. The likelihoods for the two or more medical conditions may be determined using actual and/or hypothetical test results, as discussed above. This may, for example, be useful in helping a clinician or other user determine which test or tests may be most informative in assessing both medical conditions. In addition, or alternately, a computerized system may automatically assess the likelihood that a patient has multiple medical conditions based on tests and test results. As an example, tests and test results may be provided to the computerized system, which may determine a composite likelihood for multiple medical conditions using an iterative Bayesian analysis of the tests and test results. Such assessment may include medical conditions that were not considered or otherwise indicated by a user. Using such an approach, the system may assess the likelihood for 10's, 100's or 1000's of possible medical conditions based on a same set of tests and test results. Possible medical conditions which have a composite likelihood above or below a threshold may be identified and displayed to a user, e.g., as a suggested medical condition for consideration or other investigation by a user. This may help a clinician assess which of multiple medical conditions a patient may have.

In one embodiment, a method for providing information regarding a medical condition likelihood for a patient based on multiple test results may be performed by a computerized system, e.g., a programmed computer system including instructions to perform steps of the method. First information regarding a first test regarding the patient may be received, e.g., by a user entering the first information using a computer-implemented user interface, by a computer system accessing stored first information in a database, etc. The first information may include a pre-test likelihood of the medical condition, a sensitivity for the first test with respect to the medical condition, a specificity for the first test with respect to the medical condition, and a first test result for the first test regarding the medical condition. The pre-test likelihood may be determined in different ways, such as by a user providing an estimate of the pre-test likelihood that the patient has the medical condition (e.g., based on an evaluation and/or medical history of the patient), based on epidemiological data of medical condition incidence in a relevant population, etc. In some cases, the pre-test likelihood may be a post-test likelihood determined using a Bayesian analysis of information from one or more other tests, since the method may include determining a composite likelihood based on three or more tests and test results. A first post-test likelihood of the medical condition may be determined based on the first information and using a Bayesian analysis of the first information. Second information regarding a second test regarding the patient may be received, and similar to the first information, the second information may include a sensitivity for the second test with respect to the medical condition, a specificity for the second test with respect to the medical condition, and a second test result for the second test regarding the medical condition. A second post-test likelihood of the medical condition may be determined based on the second information using a Bayesian analysis of the second information and using the determined first post-test likelihood for a second pre-test likelihood of the medical condition in the Bayesian analysis of the second information. Thus, the second post-test likelihood is determined using an iterative approach where the post-test likelihood from a prior analysis is used as a pre-test likelihood for the current analysis. The second post-test likelihood may display to a user as a composite likelihood that the patient has the medical condition based on the first and second tests, e.g., on a graphical user interface of a computer system.

As noted above, the method, or system adapted to perform the method, may be employed to assess the potential value of one or more hypothetical tests in the assessment of whether a patient has a particular medical condition. For example, in the method above, the first test may be an actual test and the first test result is a test result that resulted from actually performing the first test with respect to the patient. On the other hand, the second test may be a hypothetical test that may be performed with respect to the patient and the second test result is a selected test result from multiple possible test results for the hypothetical test. Using this information, the composite likelihood for the medical condition may be determined based on different possible test results for a hypothetical test. As an example, the composite likelihood may be determined for a selected “negative” hypothetical test result, as well as for a selected “positive” hypothetical test result, and the two composite likelihoods for the two different test results compared to each other, and/or to a suitable threshold. Based on the resulting composite likelihood(s), a determination may be made whether to actually perform the hypothetical test with respect to the patient. As an example, if a “positive” hypothetical test result would result in a composite post-test likelihood that there is a 95% probability that the patient has the medical condition, a determination may be made that the hypothetical test should be done. An even more (or less) compelling case may be made if the composite likelihoods for two different hypothetical test results differ from each other by more (or less) than a threshold. Using the example above, if a “negative” hypothetical test result would result in a composite post-test likelihood that there is a 5% probability that the patient has the medical condition, a determination may be made to perform the hypothetical test. On the other hand, if the “negative” hypothetical test result would result in a composite likelihood that there is a 93% probability that the patient has the medical condition, a determination may be made that the hypothetical test will not provide compelling enough information because the difference between the 95% and 93% probabilities is too small to justify performing the test. A computerized system may determine to have a hypothetical test performed based on the analysis of the composite likelihood(s), e.g., tests may be ordered where a difference between composite likelihoods for two different test results is more than a threshold.

In some particularly advantageous embodiments, two or more medical conditions may be assessed based on a single set of test results. This may allow a user to assess several different medical conditions which may be simultaneously present in the patient. Thus, the method described above may be extended for a second medical condition with the following:

Receiving third information regarding the first test regarding the patient, where the third information includes a pre-test likelihood of a second medical condition that is different from the first medical condition, a sensitivity for the first test with respect to the second medical condition, a specificity for the first test with respect to the second medical condition, and a third test result for the first test regarding the second medical condition. The test result for the first test is referred to as a “third” test result because the test result for the first test regarding the second medical condition may be different than that for the first medical condition. A first post-test likelihood of the second medical condition may be determined based on the third information and using a Bayesian analysis of the third information. Next, fourth information regarding a second test regarding the patient may be received (of course, this information may be received before the first post-test likelihood is determined), where the fourth information includes a sensitivity for the second test with respect to the second medical condition, a specificity for the second test with respect to the second medical condition, and a fourth test result for the second test regarding the second medical condition. A second post-test likelihood of the second medical condition may be determined based on the fourth information using a Bayesian analysis of the fourth information and using the determined first post-test likelihood of the second medical condition for a second pre-test likelihood of the second medical condition in the Bayesian analysis of the fourth information. The second post-test likelihood of the second medical condition may be displayed as a composite likelihood that the patient has the second medical condition based on the first and second tests, with the second post-test likelihood of the second medical condition being displayed simultaneously with the second post-test likelihood of the first medical condition. Simultaneous display may allow a user to more easily compare or otherwise assess the composite likelihoods for the two medical conditions. While this example involves two medical conditions, three or more medical conditions may be assessed and composite likelihoods determined displayed simultaneously for all medical conditions.

As in the example above, when analyzing two or more medical conditions, one or more hypothetical tests and selected test results may be assessed as well. Thus, composite likelihoods may be determined and displayed for multiple medical conditions for one or more hypothetical tests. Similarly, at least one of the medical conditions may be a possible medical condition that is analyzed using test results and stored information regarding multiple medical conditions and hypothetical test result information. In addition to displaying composite likelihoods, other information may be displayed such as sensitivities and specificities for the first and second (and other) tests for each of the first and second (or more) medical conditions.

In some embodiments, the method and/or a system adapted to perform steps of the method may be employed to identify one or more tests that should be performed with respect to a patient, or at least that would provide useful information regarding one or more medical conditions. For example, a plurality of post-test likelihoods may be determined for a plurality of medical conditions based on information for a plurality of different hypothetical tests using a Bayesian analysis of the information and using the determined second post-test likelihood for a pre-test likelihood in the Bayesian analysis. The information used in the Bayesian analysis for each of the plurality of different tests for each medical condition may include a sensitivity for each test with respect to each medical condition, a specificity for each test with respect to each medical condition, and a hypothetical test result for each test regarding the medical condition. One or more of the plurality of different tests may be identified and displayed as a recommended test to be performed with respect to the patient based on the post-test likelihood determined for the one or more of the plurality of different tests. As an example, tests that have a corresponding post-test likelihood that is either below a low threshold (e.g., 10%) or above a high threshold (e.g., 90%) may be identified as a recommended test. As an alternative, tests that have a difference between composite likelihoods which is greater than a threshold (e.g., 50%) for different test results may be recommended for performance.

In some embodiments, the method and/or a system adapted to perform steps of the method may be employed to identify one or more possible medical conditions that a patient is likely to have (or not have) based on a set of test results. As discussed above, this may allow a system to determine and identify one or more possible medical conditions as being likely or unlikely to be present, even where a clinician has not previously considered the medical conditions. As an example, composite post-test likelihoods for a plurality of possible medical conditions may be determined based on first and second (or more) tests and corresponding sensitivity, specificity and test result information for the first and second (or more) tests with respect to each of the possible medical conditions. Each of the composite post-test likelihoods for the plurality of possible medical conditions may be determined by:
receiving information regarding the first test regarding the patient, the information including a pre-test likelihood of the possible medical condition, a sensitivity for the first test with respect to the possible medical condition, a specificity for the first test with respect to the possible medical condition, and a test result for the first test regarding the possible medical condition;
determining a post-test likelihood of the possible medical condition based on the information and using a Bayesian analysis of the information;
receiving information regarding the second test regarding the patient, the information including a sensitivity for the second test with respect to the possible medical condition, a specificity for the second test with respect to the possible medical condition, and a test result for the second test regarding the possible medical condition; and
determining a composite post-test likelihood of the medical condition based on the information regarding the second test using a Bayesian analysis of the information and using the determined post-test likelihood for a pre-test likelihood of the possible medical condition in the Bayesian analysis of the information regarding the second test.

Although only two tests are indicated above, the process for determine each composite post-test likelihood may involve analysis using three or more tests and corresponding test results, including test and test results that are hypothetical. One or more of the possible medical conditions may be displayed as a suggested medical condition for investigation based on the composite post-test likelihood for each of the one or more possible medical conditions. For example, automated analysis of a set of test results for a patient may result in one or more medical conditions having a composite post-test likelihood in excess of 90%. These medical conditions and corresponding composite likelihoods may be displayed to a user, e.g., as a suggestion that the patient has the displayed medical conditions.

As noted above, a computerized system may be adapted to perform various steps including receiving information regarding tests and test results, determining post-test likelihoods for different medical conditions based on test information, displaying post-test likelihood information for multiple tests for a medical condition, etc. In some embodiments, at least some test information may be obtained from a computer database that stores test information for multiple tests and for multiple medical conditions. For example, a computer database may store sensitivity and specificity information for multiple tests regarding multiple medical conditions, as well as test result information for each of the tests and regarding each of the multiple medical conditions. This information may be accessed by the computerized system to perform the analyses discussed herein, e.g., for determining post-test likelihood for medical conditions based on actual or hypothetical test results. The database information may be obtained from various sources, such as clinical study data, from a user, and/or from past analysis data from determining composite likelihood information using Bayesian analysis. For example, data may be stored for each analysis performed by the system for each patient, the patient's test information, composite likelihood information, and ultimate diagnosis information for the patient. This stored data may be used to generate or refine sensitivity and/or specificity information for tests for medical conditions, to generate or refine pre-test likelihood information for medical conditions, and other. This generated or refined data may then be used in future analysis involving Bayesian assessment of tests and test results for other patients.

In addition, or alternately, stored data regarding past assessment may be used in other ways, such as analyzing and reporting on clinician test orders, medical condition diagnosis, treatment, and other activity. For example, reports may be generated regarding an average number of tests ordered by a clinician or group of clinicians at a facility in relation to one or more medical conditions. Information for a clinician or group of clinicians may be compared to information for other persons or groups, or to a standard, to assess whether an appropriate number or type of tests are being employed to diagnose particular conditions. Reports may be generated regarding accuracy of initial medical condition diagnosis as compared to final diagnosis, e.g., to identify areas where a clinician or group of clinicians may need additional training or information. In other cases, reports may be generated regarding the usefulness of particular tests, either in relation to particular medical conditions or on the whole. Tests that generally provide less useful information may be identified as less favored and used less frequently.

In some embodiments, a likelihood of a medical condition that has multiple potential causes and/or sub-conditions may be assessed, e.g., using one or more tests that has different results regarding the medical condition, a cause of the medical condition and/or sub-condition of the medical condition. For example, an assessment may be made not only whether a medical condition is present, but also one or more causes of the medical condition, based on one or more tests that may provide information regarding the presence of a cause, and/or whether a sub-condition of the medical condition is present. For example, a computerized system may receive first information regarding a first test regarding the patient, with the first test for detecting whether a first cause or sub-condition of the medical condition is present. The first information may include a first pre-test likelihood of the medical condition, a sensitivity for the first test with respect to the medical condition, a specificity for the first test with respect to the medical condition, and a first test result for the first test regarding the first cause. A first post-test likelihood of the medical condition may be determined based on the first information and using a Bayesian analysis of the first information, e.g., using the techniques described above. The first post-test likelihood may indicate a likelihood that the medical condition is present and/or indicate a likelihood that the first cause of the medical condition is present. Second information regarding a second test regarding the patient may be received, with the second test detecting whether a second cause or sub-condition of the medical condition is present. The second information may include a second pre-test likelihood of the medical condition, a sensitivity for the second test with respect to the medical condition, a specificity for the second test with respect to the medical condition, and a second test result for the second test regarding the second cause. A second post-test likelihood of the medical condition may be determined based on the second information and using a Bayesian analysis of the second information. Like the first post-test likelihood, the second post-test likelihood may indicate a likelihood that the medical condition is present and/or indicate a likelihood that the second cause or sub-condition of the medical condition is present. For example, the sensitivity and specificity for the first test with respect to the medical condition may be a sensitivity and specificity for the first test regarding whether the first cause is present, and the sensitivity and specificity for the second test with respect to the medical condition may be a sensitivity and specificity for the second test regarding whether the second cause is present. Thus, the first and second post-test likelihoods may represent a likelihood that the first and second causes are present. In some cases, the first and second tests may be a same test and the first and second test results are results regarding the presence of the first and second cause, respectively. That is, one particular test (such as a bacterial and fungus culture) may be referred to herein as “first and second” tests or different tests because the test provides different results for different medical conditions, causes and/or sub-conditions (e.g., the bacterial and fungus culture will provide different results regarding whether one or more bacterial strains is present, whether a fungus is present, and even whether a virus is present—the test will return no results or a negative result for a virus) even though a single test is performed. The first and second pre-test likelihoods may each be a respective likelihood that the first cause and the second cause are the cause of the medical condition, and in such a case the first and second pre-test likelihoods may be a fractional portion of a pre-test likelihood for the medical condition. For example, if a pre-test likelihood for the medical condition is 20%, and clinical data shows that the first cause is present in 10% and the second cause is present in 20% of cases in which the medical condition is diagnosed, the first pre-test likelihood may be 2% and the second pre-test likelihood may be 4%.

The system may receive third information regarding a third test regarding the patient, with the third test for detecting whether a result of the medical condition is present. The third information may include a sensitivity for the third test with respect to the medical condition, a specificity for the third test with respect to the medical condition, and a third test result for the third test regarding the medical condition. Again, note that the third test may actually be the same test as that for the first and second tests, but the test provides a test result for the presence of the medical condition itself, in addition to providing a test result regarding whether the first and/or second cause is present. Of course, the first, second and/or third tests may be completely different tests. A third post-test likelihood of the medical condition may be determined based on the third information using a Bayesian analysis of the third information and using a union of the determined first post-test likelihood and the determined second post-test likelihood for a third pre-test likelihood of the medical condition in the Bayesian analysis of the third information. Details regarding how such a union is determined are provided below, but the union may be required because the first and second post-test likelihoods represent the presence of the first and second causes or sub-conditions, respectively, rather than the medical condition itself. The third post-test likelihood may be displayed as a composite likelihood that the patient has the medical condition based on the first, second and third tests, and the third post-test likelihood may be displayed simultaneously with the first and second post-test likelihoods, e.g., to allow a clinician the ability to assess a potential cause or sub-condition of the medical condition as well. Such information may be useful for treatment.

An assessment like that above may be performed in a different order, e.g., test results regarding the presence of a medical condition itself may be assess to determine a post-test likelihood of the medical condition, and thereafter test results (from a same or different set of tests) for one or more causes and/or sub-conditions may be assessed to determine not only a post-test likelihood for the medical condition, but also a post-test likelihood that one or more causes and/or sub-conditions may be present. For example, first information may be received by a computerized system regarding a first test regarding the patient, with the first test for detecting whether a result of the medical condition is present. The first information may include a pre-test likelihood of the medical condition, a sensitivity for the first test with respect to the medical condition, a specificity for the first test with respect to the medical condition, and a first test result for the first regarding the medical condition. A first post-test likelihood of the medical condition may be determined based on the first information using a Bayesian analysis of the first information. Thereafter, second information regarding a second test regarding the patient may be received with the second test for detecting whether a second cause or sub-condition of the medical condition is present. The second information may include a second pre-test likelihood of the medical condition (such as an incidence rate of the second cause or sub-condition in connection with the medical condition used to divide the first post-test likelihood into a corresponding fractional portion used in the Bayesian analysis), a sensitivity and specificity for the second test with respect to the medical condition (such as whether the second cause is present), and a second test result for the second test regarding the second cause. A second post-test likelihood of the medical condition may be determined based on the second information and using a Bayesian analysis of the second information, and the second post-test likelihood may represent the likelihood that the second cause is the cause of the medical condition, or that the second sub-condition is present. Similarly, third information regarding a third test regarding the patient may be receive with the third test for detecting whether a third cause of the medical condition is present. The third information may include a third pre-test likelihood of the medical condition (such as an incidence rate of the third cause or sub-condition in connection with the medical condition used to divide first post-test likelihood into a corresponding fractional portion used in the Bayesian analysis), a sensitivity and specificity for the third test with respect to the medical condition (such as whether the third cause is present), and a third test result for the third test regarding the third cause or sub-condition. A third post-test likelihood of the medical condition may be determined based on the third information and using a Bayesian analysis of the third information, and the third post-test likelihood may represent the likelihood that the third cause is the cause of the medical condition, or that the third sub-condition is present. A fourth post-test likelihood of the medical condition may be determined based on a union of the determined second post-test likelihood and the determined third post-test likelihood, and the fourth post-test likelihood may be displayed as a composite likelihood that the patient has the medical condition based on the first, second and third tests.

These and other aspects of the invention will be apparent from the following description and claims.

### BRIEF DESCRIPTION OF THE DRAWINGS

Aspects of the invention are described below with reference to illustrative embodiments, some features of which are illustrated in the following figures.

FIG. 1 shows a graphical user interface for receiving and/or displaying information regarding one or more tests with respect to a medical conditions;

FIG. 2 shows a graphical user interface for receiving and/or displaying information regarding one or more tests with respect to one or more medical conditions;

FIG. 3 shows the graphical user interface of FIG. 2 including test results and other information for two medical conditions;

FIG. 4 show another graphical user interface for receiving and/or displaying information regarding one or more tests with respect to a medical conditions;

FIG. 5 shows the graphical user interface of FIG. 4 configured to identify information regarding one or more medical conditions for assessment;

FIG. 6 shows the graphical user interface of FIG. 4 configured to identify information regarding pre-test likelihoods for one or more medical conditions to be assessed;

FIG. 7 shows the graphical user interface of FIG. 4 configured to identify test information for medical conditions being assessed;

FIG. 8 shows the graphical user interface of FIG. 4 configured to identify test and test result information;

FIG. 9 shows the graphical user interface of FIG. 4 configured to identify test result and specificity and sensitivity information;

FIG. 10 shows the graphical user interface of FIG. 4 revised to include additional test and test result information along with determined post-test likelihood information using a Bayesian analysis of the information;

FIG. 11 shows the graphical user interface of FIG. 4 configured to identify hypothetical test and test result information for determining a post-test likelihood based on the hypothetical information;

FIG. 12 shows the FIG. 11 graphical user interface including modified hypothetical test results and corresponding post-test likelihood information;

FIG. 13 shows the FIG. 11 graphical user interface including modified pre-test likelihood information and corresponding post-test likelihood information;

FIG. 14 shows the FIG. 11 graphical user interface including modified pre-test likelihood information and corresponding post-test likelihood information;

FIG. 15 shows a graphical user interface including multiple hypothetical tests and corresponding composite likelihoods for different test results;

FIG. 16 shows the graphical user interface of FIG. 15 with the hypothetical tests listed according to descending positive test result composite likelihood;

FIG. 17 shows the graphical user interface of FIG. 15 with the hypothetical tests listed according to ascending negative test result composite likelihood;

FIG. 18 shows a graphical user interface including two medical conditions and corresponding hypothetical tests and selectable test results;

FIG. 19 shows a graphical user interface including a medical condition, a set of hypothetical tests and test results, and an additional hypothetical test and test results;

FIG. 20 shows a graphical user interface including a medical condition, multiple tests for results of the medical condition, and multiple potential causes for the medical condition;

FIG. 21 shows the graphical user interface of FIG. 20 including a test result for one of the potential causes of the medical condition;

FIG. 22 shows the graphical user interface of FIG. 20 including test results for three of the potential causes of the medical condition;

FIG. 23 shows an illustrative graphical display of post-test likelihood for a medical condition for multiple tests and different test results; and

FIG. 24 shows the graphical user interface of FIG. 1 including test result information and resulting post-test likelihoods determined in accordance with inventive embodiments.

### DETAILED DESCRIPTION

It should be understood that aspects of the invention are described herein with reference to certain illustrative embodiments and the figures. The illustrative embodiments described herein are not necessarily intended to show all aspects of the invention, but rather are used to describe a few illustrative embodiments. Thus, aspects of the invention are not intended to be construed narrowly in view of the illustrative embodiments. In addition, it should be understood that aspects of the invention may be used alone or in any suitable combination with other aspects of the invention.

As described above, embodiments that include features of the invention provide the ability to determine and display to a user the likelihood that a patient has one or more potential medical conditions based on one or more diagnostic or other tests. (As used herein, a “patient” includes any animal, whether human or otherwise. Thus, aspects of the invention may be employed for humans, horses, dogs, etc.) The likelihood that the patient has a particular medical condition is determined based on an iterative Bayesian analysis of test results as well as sensitivity and specificity information for the tests regarding the medical condition. As described in more detail below, the iterative analysis involves using the post-test likelihood determined for a test result, and then using the determined post-test likelihood as a pre-test likelihood for a subsequent test result analysis. Thus, a composite likelihood may be determined for multiple tests by performing a Bayesian analysis for a first test result using a pre-test likelihood (e.g., estimated by a clinician) which determines a first post-test likelihood, then performing a Bayesian analysis for a second test result using the first post-test likelihood to determine a second post-test likelihood, and so on. The composite likelihood, i.e., a last post-test likelihood determined for a set of test results, may be displayed in various forms, such as a percentage chance that the patient has the medical condition, a percentage chance that the patient does not have the medical condition, the odds that the patient has, or does not have, the medical condition, and others. For ease of reference, all such types of indication are referred to as a likelihood that the patient has a medical condition, or more simply, the likelihood of the medical condition. The medical condition(s) that are evaluated may be any suitable medical condition, such as a disease, injury, disability, disorder, state, syndrome or other. (Herein, the term “disease” is often used rather than “medical condition.” However, such use of “disease” should not be interpreted as limiting the scope of any invention in any way.) Medical conditions to be assessed may be selected in any suitable way, such as at random, by a user (such as a clinician or other health care professional), based on tests performed and/or test results (e.g., a battery of tests may be associated with one or more medical conditions, which are automatically selected for assessment when that battery of tests is performed), based on symptoms that a patient indicates and/or exhibits and/or are observed by a clinician or in another way (such as by one or more sensors detecting features of a patient), and so on. Tests may take a variety of different forms, such as diagnostic tests performed by a laboratory and/or using specialized equipment and/or techniques (such as nucleic acid sequencing, blood testing, vital sign detection whether manually or using electronic devices, biomarker identification, pathology results, MRI or other scans or imaging and results of analysis of such imaging, etc.) Tests may be performed in other ways, such as by a clinician or other person observing a patient or patient state, such as a patient's physical appearance, response to physical probing or other contact, a patient's ability to perform physically, and so on. Thus, tests and test results may be performed using objective and/or subjective measures, and test results may be described or otherwise provided in any suitable way, such as on a numeric scale, as positive or negative value, as a color selected from two or more colors, as a word selected from a group of words, etc. Thus, a test may have multiple possible test results, which may be characterized in a variety of different ways.

While embodiments described below generally relate to a single patient, features of the invention may be employed with multiple patients, whether simultaneously or separately. In some embodiments, information used to assess one or more medical conditions for a patient as well as results of analysis may be combined with patient tracking, whereby a patient identifier is associated with the information, e.g., for storage in and later retrieval and use from a computer system. Electronic storage of such information (e.g., including medical conditions evaluated, likelihoods of the medical condition(s) for various tests or test combinations, etc.) allow for a variety of benefits, such as including medical condition likelihood in the patients' charts and/or other records, the ability to recall patient information to allow review of prior testing and test results; the addition of any new tests and test results received for subsequent Bayesian analysis; and the gathering, over time, of statistics related to the analysis of past patients. For example, patient tracking allows a clinician to see how many past patients presenting with particular symptoms, or members of particular patient groups (grouped by sex, age, or other identifier), have reached particular final diagnoses and outcomes.

While features of the invention can be implemented by hand, e.g., with pen and paper, information may be received, analyses may be performed and information displayed using any suitable electronic device or set of devices. For example, in some embodiments, methods for performing medical condition likelihood analysis may be implemented as an application, software or other set of instructions on a mobile device (smartphone, tablet, etc.), a laptop, desktop, or server, or combinations of such devices, e.g., operating over one or more communications networks such as the Internet, cellular, LANs, WANs, etc. An application used to perform analysis may be or include a combination of several modules, and may be integrated in, or compatible with electronic medical record systems. Information may be displayed to a user in different ways, such as on a printed card or paper or other display, such as an electronic display on a user device.

In at least some embodiments, methods and/or systems that operate according to at least some features of the invention are capable of determining and displaying accurate, clinically useful likelihoods for one or more medical conditions because of an incorporation of some key rules of Bayesian statistics:
The incidence rate of the suspected disease or other medical condition and/or the sensitivity and specificity of a test with respect to the medical condition (e.g., positive and negative test results from a clinical trial from which the sensitivity and specificity were derived) are determined impartially.
Irrelevant differences in groups are ignored.
Any preferred hypothesis regarding a patient's medical condition should be assessed, drawing upon existing confidence to develop a more convincing conclusion.
Probabilities are to be rational and coherent (e.g., probabilities must sum to 100%).

Looking closer at the first point above, note that the incidence of the medical condition and the size of the studies on which the sensitivity and specificity analysis are based can be important factors. The rarer the disease, the larger the patient sample size required to accurately determine the incidence rate. An accurate estimate of disease incidence in a patient population can be important for determining its likelihood. Yet in current practice, this is usually a subjective input by the clinician—a shortcoming of clinical applications of Bayesian statistics to date. Yet changing the incidence rate from, say, 10% to 2% of the population may return dramatically different Bayesian probabilities, even though the sensitivity and specificity of the test is unchanged. In some embodiments, this significance can be accounted for, and may provide advantages.

In at least some embodiments, a novel application of a composite probability determination of multiple, iterative Bayesian analyses is employed. This approach may provide advantages over a standard Bayesian inference, which requires that all events (test results, die rolls, coin tosses, etc.) be identical and independent events. Some embodiments of the invention may be used to analyze uncertainty in the probability that a patient has a medical condition, arising from the use of imperfect, but well-studied diagnostic tests or a combination of tests. Moreover, the inventive methods and systems may provide an ability to refine processes associated with patient diagnosis, including test selection, sensitivity and/or specificity for tests for particular medical conditions, revision of pre-test likelihood for medical conditions, etc. based on the results of analysis across a large sample of patients.

Another feature of the inventive methods and/or systems is the determination of likelihoods for multiple diseases that use the same tests (or at least some of the same tests). For instance, consider a hepatic injury, which is typically ascribed to either alcohol, drug, or viral causes, and treated according to the cause assumed by patient history and test results. Certain tests for the type of hepatic injuries are sensitive and specific to at least two of the disease state causes, so the likelihood of each cause can be independently determined using a same set of test results, reducing the chance of a clinician missing or misdiagnosing the most probable cause. This is especially useful to inexperienced clinicians who may be overly reliant on marginally accurate tests and who discount the possibility of alternate disease states due to confirmation bias, non-rational result interpretation, and other commonly recognized statistical and diagnostic errors. Thus, inventive methods and systems can be especially useful in understanding diagnostic uncertainty for clinicians and other health care professionals in facilities that regularly conduct differential diagnosis, such as emergency rooms, intensive care units, general wards, and specialty clinics. Furthermore, techniques described herein may be useful as a classroom teaching tool using hypothetical patients and test results.

In at least some embodiments, a method or system may provide at least one or more of the following:

1. A computerized method and/or system by which a user can perform a Bayesian analysis to determine a likelihood for one or more medical conditions based on one or more test results.

2. A technique for determining test sensitivity and specificity from large randomized study data. This calculator also provides 95% confidence intervals of the sensitivity and specificity, so the user can understand the degree of uncertainty in the trials used to define test accuracy.

3. An ability to display medical condition likelihood based on a test result and an initial disease probability. A display for two or more tests may also be generated, incorporating two or more test results and/or two or more initial (or pre-test) probabilities for the medical condition. The medical condition likelihood for a single test can be displayed as a two-line graph with an initial probability on the X-axis and a post-test probability on the Y-axis. This display can allow for the assessment of test results for multiple pre-test probabilities, as well as different test results, e.g., an initial probability may be selected on the X-axis and the corresponding post-test probability on the Y-axis identified for a selected test result.

4. A database of diseases and associated diagnostic or other tests which can allow for easy access to suitable sensitivity, specificity and/or other data used to assess medical condition likelihood for multiple medical conditions and tests without requiring extensive research.

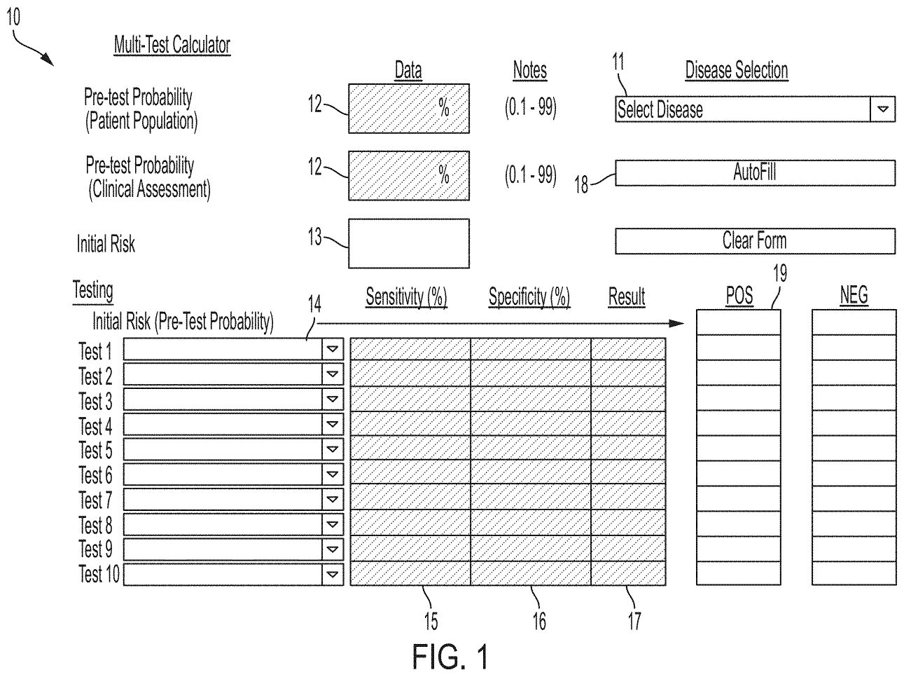

FIG. 1 shows a graphical user interface 10, e.g., implemented on a user computing device, for receiving and/or displaying information regarding one or more tests as well as a composite likelihood for medical conditions based on a Bayesian analysis of test information. While a graphical user interface employed in embodiments may be arranged in different ways as discussed more below, in this embodiment, the interface allows a user to select or otherwise identify a medical condition to be assessed for a patient at drop-down menu 11. While a user may click or otherwise select the menu 11 to cause display of one or more selectable medical conditions, the user may identify the medical condition in other ways, such as by typing the medical condition into the menu 11. The user may also provide information regarding the general incidence of the medical condition at box 12, such as by entering a percentage number, and/or information regarding a pre-test likelihood 13 that a patient has the medical condition, e.g., from a clinician's initial assessment based on examining the patient, the patient's medical history, etc. In this embodiment, the pre-test likelihood is indicated as a positive probability value (POS), e.g., a percentage that the patient has the medical condition, but could be characterized in other ways, such as by a negative probability value (NEG, e.g., a percentage that the patient does not have the medical condition), and others. The user may provide information regarding one or more tests performed with respect to the patient, e.g., diagnostic tests performed by a laboratory, using one or more drop-down menus 14. A user may click a menu 14, and one or more tests may be listed for the user to select, e.g., using a mouse or other pointing device. In some cases, the tests that are listed in the menu 14 may correspond in some way to an indicated medical condition, e.g., a set of tests that were actually performed or are typically performed in connection with the indicated medical condition may be listed or highlighted for selection. However, this is not required and a list of all possible tests may be provided with the menu 14 and/or a user may manually enter a test into the menu 14, e.g., by typing the name of the test into the menu box 14. Sensitivity and specificity information for each indicated test may be provided using boxes 15, 16, respectively. As will be appreciated and as is discussed more below, the sensitivity and specificity information for each test will correspond to the indicated medical condition, as tests may have different sensitivity and specificity values for different medical conditions. A user may provide the sensitivity and/or specificity information, e.g., by typing percentage numbers into the boxes 15, 16, or such information may be automatically entered, e.g., by clicking on the “autofill” box 18. Clicking the autofill box 18 may cause the computerized system to access sensitivity and specificity information for one or more tests regarding the indicated medical condition from a database and enter the corresponding values in the boxes 15, 16. Alternately, the sensitivity and specificity information may be retrieved and entered in response to test selection or other indication. This can eliminate any need for a user to know or otherwise determine these values.

The user may also provide test result information in box 17 which indicates the results of the test with respect to the medical condition. As described above, the test result information may be provided in different ways, such as a percentage value, a numeric value, a “positive” or “negative” indication, etc. Note that the autofill box 18 may provide the user with the ability to have information for all of the pre-test likelihood 13, test 14, sensitivity 15, specificity 16 and test result 17 information automatically populated into the graphical user interface 10. As an example, the interface 10 may allow a user to identify a particular patient, and the patient's medical or other record (e.g., stored in a database) may be accessed to retrieve information regarding tests performed, as well as test results and sensitivity and specificity information for the tests with respect to the identified medical condition. This may make the assessment of a medical condition likelihood convenient for a user, as well as help ensure that the information provided via the interface 10 is correct. In some cases, test result information 17 may need to be converted to binary form, e.g., negative or positive, for analysis. The computerized system may automatically make such conversion as needed, e.g., by employing a threshold value for numerical range test results whereby values over the threshold are assigned one binary value (e.g., positive) and values below the threshold are assigned another binary value (e.g., negative). Such threshold or other information used to convert test results to binary or other useful values for use in Bayesian analysis may be stored in a database, e.g., along with specificity and sensitivity information for the corresponding test for multiple medical conditions, and may be retrieved and used for test result conversion as needed. A user could make such conversion as well, and different threshold values may be employed for different tests and medical conditions.

Using the pre-test likelihood information 13, as well as the sensitivity and specificity information 15, 16 and test result information 17, an iterative Bayesian analysis may be performed to determine a post-test likelihood that the patient has the indicated medical condition. The determined post-test likelihood may be displayed, for example, in the boxes of column 19. In this example, the uppermost box in column 19 includes the pre-test likelihood 13, which is used to determine the post-test likelihood for Test 1 (or the first test listed in the boxes 14). The post-test likelihood determined based on Test 1 is displayed in the corresponding box in column 19, and then is used as a pre-test likelihood for determining a post-test likelihood based on information for Test 2, and so on until a post-test likelihood is determined using all test information. The last post-test likelihood determined may be displayed in the lowermost box of column 19 or in other ways, and may represent the composite likelihood that the patient has the medical condition based on the test information. A user may assess the post-test likelihood and use it as a factor in determining whether the patient has the medical condition and/or for other purposes.

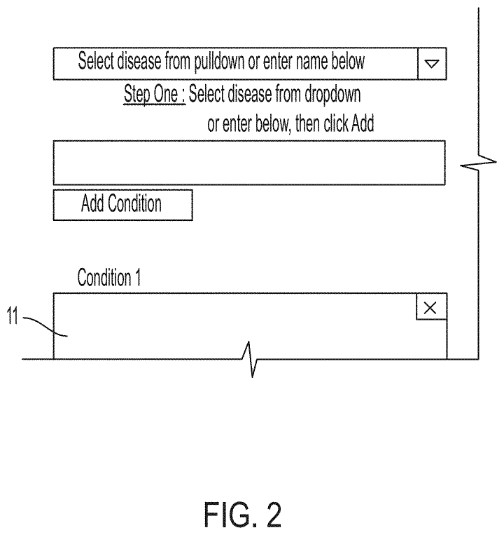

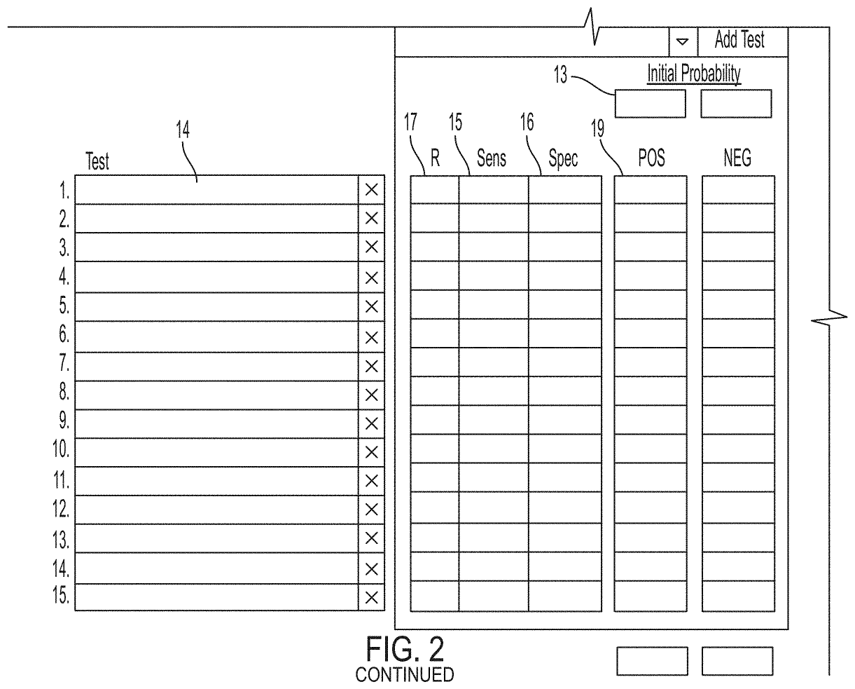

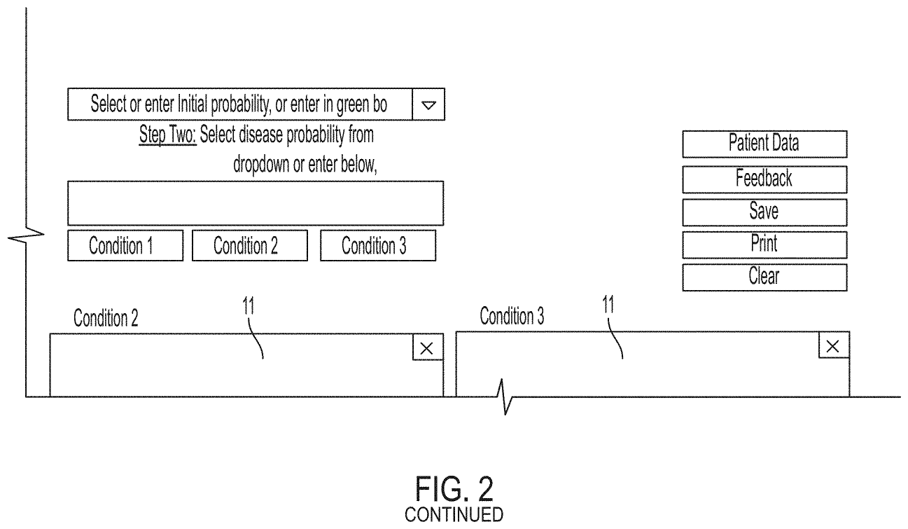

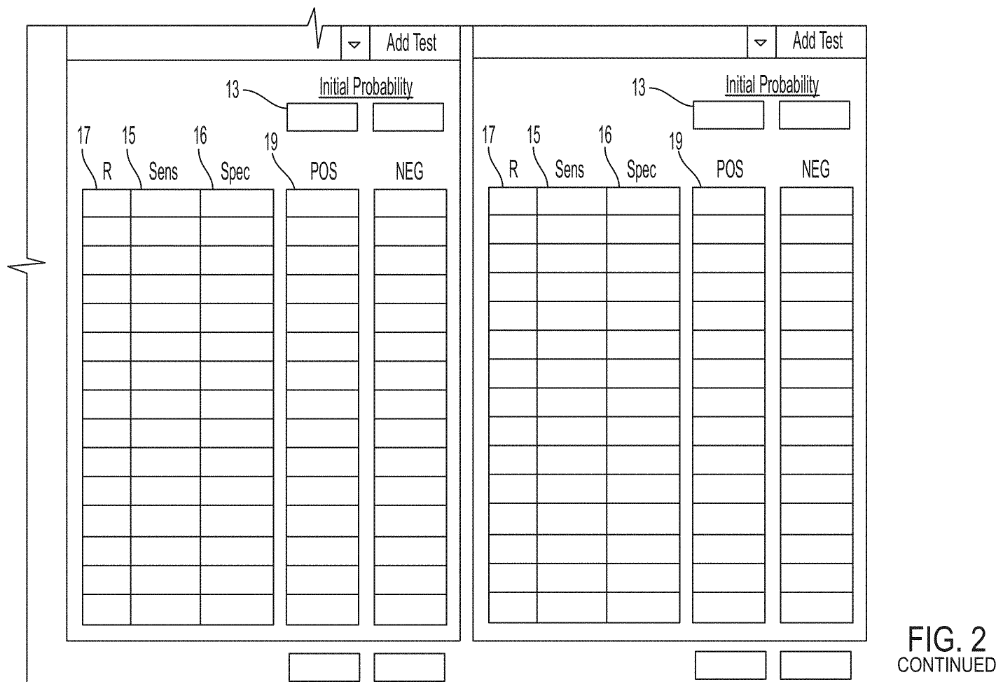

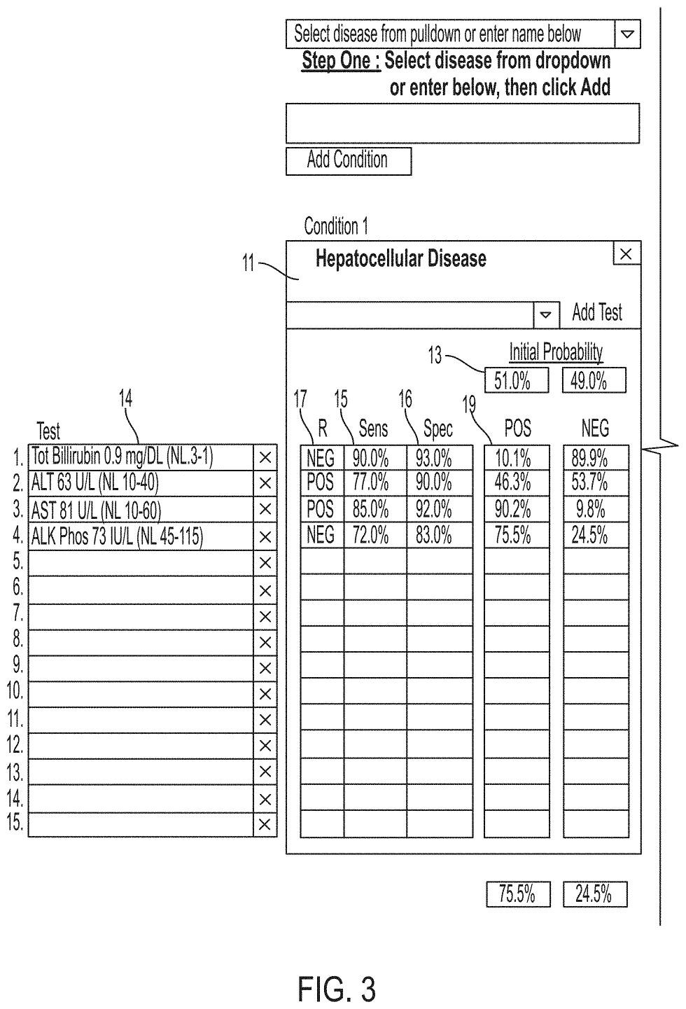

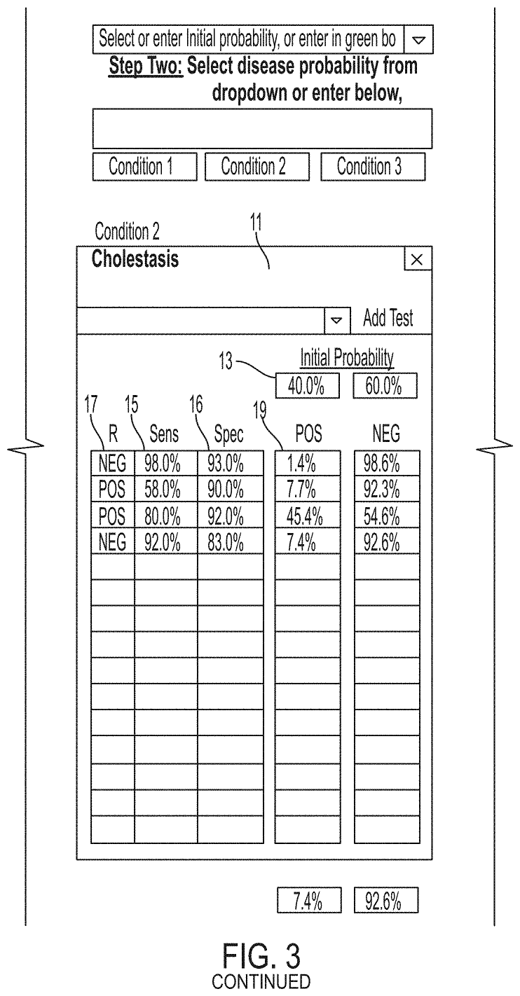

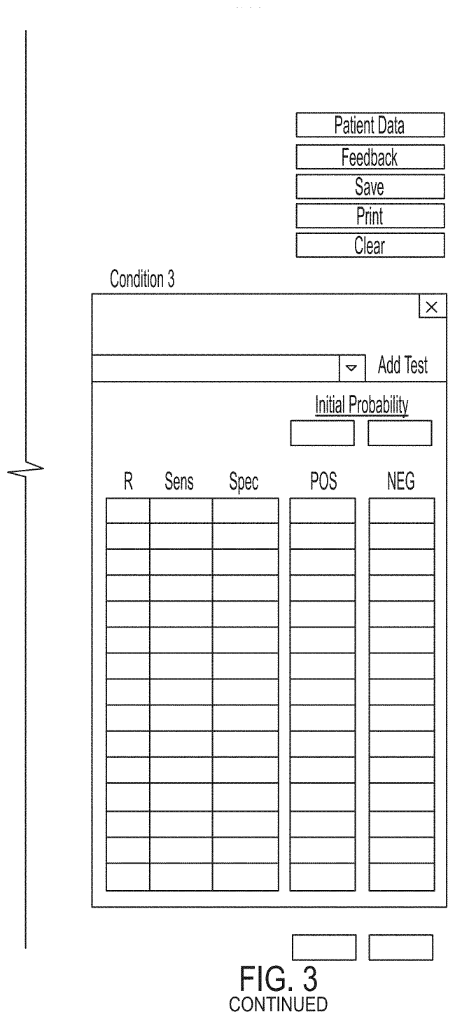

While the graphical user interface 10 of FIG. 1 shows an assessment performed for a single medical condition, the interface 10 may include two or more medical conditions, e.g., as shown in FIG. 2. In the example of FIG. 2, three medical conditions 11 (Diseases 1-3) are displayed along with corresponding pre-test likelihood information 13, sensitivity 15, specificity 16, and post-test likelihood information 19. Note that multiple tests 14 may be indicated along with corresponding test results 17. In this example, the test results 17 are the same for each medical condition 11, but in other embodiments test results 17 may be different for different corresponding medical conditions 11. As one example, a particular test may provide a positive result for one medical condition, while providing negative or irrelevant results for another medical condition. In such a case, the test results 17 may be indicated for each corresponding medical condition 11, e.g., in a way similar to that in FIG. 1. FIG. 3 shows an example display of an interface 10 that includes two medical conditions 11 indicated and post-test likelihood information 19 determined based on test results 17 for four tests. In this example, a determination that there is a 7% likelihood that the patient has the medical condition “choleostasis” and a determination that there is a 76% likelihood that the patient has the medical condition “hepato cellular disease” is indicated for the test result information. Based on this information, a user may determine it is significantly less likely that the patient has medical condition “choleostasis” than “hepato cellular disease.” A method for using a Bayesian analysis to determine a post-test likelihood that a patient has a medical condition based on results of two or more tests, i.e., a composite likelihood of the medical condition, is as follows.

Bayes' Theorem is shown below where P=probability, X is a multiplication, A is an event, and B is a condition, read “A given B”:

P(A|B)=(P(B|A)XP(A))/P(B)

Expanding Bayes' Theorem for a binary discrete event regarding the probability that disease is present given a positive test results gives the following, where positive (+) indicates where a test result is positive, negative (−) indicates where a test result is negative, and “diseased” or “absent” indicates the medical condition is present or absent, respectively:

P(diseased|+)=(P(diseased|+)XP(diseased))/(P(+|diseased)XP(diseased)+P(+|absent)XP(absent))

In addition to the result shown above regarding the probability that disease is present given a positive test result, there are three other possibilities in outcomes: disease present and a negative test result, disease absent and a positive test result, and disease absent and a negative test result. These are shown below:

P(absent|−)=(P(absent|−)XP(absent))/(P(−|absent)XP(absent)+P(−|diseased)XP(diseased))

P(absent|+)=1−P(diseased|+)

P(diseased|−)=1−P(absent|−)

Going back to the initial nomenclature of P(A|B)=(P(B|A) X P(A))/P(B), and defining the probability of event B and “not B” as summing to 100%, we can now continue past the standard usage of Bayes' Theorem to a looping or iterative formula that is quite different in the expected error and is not constrained by the rules of a Bayesian Inference (that requires all tests be identical). Therefore, instead of the typical method of a composite probability of dual conditions such as P(A|B and C) using Bayesian Inference or posterior weighted mean probabilities, where we have:

Posterior mean=Prior weight X Prior mean+Data weight X Data mean

we can instead say, where a first test (Test 1) has a positive result indicated as +1, and a second test (Test 2) has a positive result indicated as +2, etc., that:

P(diseased |+1 and +2)=(P(diseased|+1)XP(diseased|+2))/(P(+|diseased)XP(diseased|+2)+P(+|absent)XP(absent|+2))

whereby:

P(diseased|+2)=(P(+2|diseased)XP(diseased))/P(−2)

and, from above:

P(absent|+2)=1−P(diseased|+2)

This method creates a loop that is an iteration that can be repeated multiple times for multiple tests, without increasing, linearly or otherwise, the error from the initial pre-test likelihood estimate. Also eliminated is any need to weight non-identical tests. The resulting post-test likelihood is also rational, coherent, and independent of order (e.g., changing the order in which tests are assessed such as changing “given +1 and +2” to “given +2 and +1” above, has no effect on the resulting post-test likelihood determined). In the above example, since two tests are considered instead of one and there are two possible initial conditions (diseased or absent disease), there are eight possible outcomes, but all eight outcomes can be worked out similarly to the one above, for instance “diseased, given +1 and −2,” and so on.

This iterative approach to determining a post-test likelihood may provide advantages over other probabilistic techniques. As discussed above, FIG. 3 shows data for a situation regarding a liver disease patient. In liver disease, a liver panel blood test is normally ordered, with a variety of test results included that indicate normal or abnormal liver function. Depending on the suspected injury and cause of the injury, the indicators from the blood test are not produced as “positive” or “negative,” but rather within a prescribed normal range (WNL) or abnormal range (ABN—above or below the normal range). For specific injuries, the exact amounts of abnormal values of certain markers are important, but for the point of this example we will assume that both hepatocellular disease and cholestasis have identical abnormal ranges, and that all abnormal ranges are elevated above the normal range of levels that would exclude other possible liver diseases.

For our hypothetical patient, the following tests are conducted: total bilirubin, alanine aminotransferase (ALT), aspartate aminotransferase (AST), and alkaline phosphatase (ALK Phos) as shown in FIG. 3. We will consider test results within the normal range as negative for both hepatocellular disease and cholestasis, and elevated results as positive.

In this example, total bilirubin and ALK Phos were found to be normal, but ALT and AST were elevated. If the tests had all agreed (all normal/negative or all elevated/positive), the diagnosis would be simplified and a Bayesian analysis would provide little benefit. But in this case, the clinician is unsure of how to interpret these results. The clinician's original assumption of hepatocellular disease was based on the patient's presentation, and an initial pre-test likelihood 13 was estimated at 51% probability for the disease. The sensitivities and specificities of the four tests for hepatocellular disease are shown in Table 1 below (as well as in FIG. 3):

TABLE 1

| Test for | | | |
|---|---|---|---|
| Hepatocellular | | | |
| Disease | Sensitivity | Specificity | Source |
| Total Bilirubin | 90% | 93% | Dig Dis Sci. 1983 |
| | | | February; 28(2):129-36 |
| ALT | 77% | 90% | Dig Dis Sci. 1983 |
| | | | February; 28(2):129-36 |
| AST | 85% | 92% | Dig Dis Sci. 1983 |
| | | | February; 28(2):129-36 |
| ALK Phos | 72% | 83% | Dig Dis Sci. 1983 |
| | | | February; 28(2):129-36 |

Now, consider if the disease probability were analyzed using a simple Bayesian calculator where each test is analyzed individually. Table 2 shows the results of disease probability determined using this technique:

TABLE 2

| Test for Hepatocellular | | Initial | Post-Test |
|---|---|---|---|
| Disease | Result | Probability | Probability |
| Total Bilirubin | NEG | 51% | 10% |
| ALT | POS | 51% | 89% |
| AST | POS | 51% | 92% |
| ALK Phos | NEG | 51% | 26% |

With four individual Bayesian calculations, four different results are determined. Two (positive) results from the ALT and AST tests yield a probability that the patient has hepatocellular disease of 89% and 92%, respectively. However, the other two results from total bilirubin and ALK Phos give a probability of 10% and 26% respectively. The overall probability of the patient having hepatocellular disease is indeterminate with two tests giving a relatively high probability, and two giving a relatively low probability. This information in Table 2 is contrasted with the iterative Bayesian analysis performed according to embodiments of the invention, the results of which are shown in FIG. 3. In FIG. 3, a composite post-test likelihood for hepatocellular disease for all four tests is shown to be about 76% (75.5%). This composite likelihood based on multiple tests provides a user with much more easily understood information, and takes all test results and their corresponding sensitivities and specificities into account. As those of skill will appreciate, the composite post-test likelihood determined using the iterative Bayesian analysis will be the same regardless of the order in which the tests are listed in FIG. 3 and are analyzed. For example, the four tests in FIG. 3 could be listed in any order and the composite post-test likelihood will remain the same at about 76%. This can provide a powerful advantage for the inventive system, since tests may be performed and/or analyzed in any order without affecting the post-test composite likelihood (provided, of course, that the test results are the same). It should also be noted that while four tests are shown in this example, any number of tests may be assessed, and assessment of any number of tests may provide useful results. For example, as shown in FIG. 3, the post-test composite likelihood 19 for hepatocellular disease for the first three tests listed is about 90%, and is about 45% for cholestasis. These results may suggest that it is more likely that hepatocellular disease is present than cholestasis, but that additional testing may be required. As a result, the fourth test listed may be ordered, and its assessment confirms that the likelihood of hepatocellular disease is more likely, maybe much more likely, to be present than cholestasis.

FIG. 3 also illustrates another advantage provided by inventive embodiments, i.e., that multiple medical conditions can be assessed and information for all medical conditions displayed simultaneously, and/or that multiple medical conditions can be assessed using a common set of tests and test results. For example, assume that the user in FIG. 3 initially assessed only the likelihood of hepatocellular disease, but not cholestasis. After assessing hepatocellular disease, the user may decide to investigate possible alternative diagnoses other than hepatocellular disease and realizes that all four test results used to assess hepatocellular disease can also be used to assess the likelihood of cholestasis. The sensitivities and specificities of these tests for cholestasis are shown in Table 3 below, as well as in FIG. 3:

TABLE 3

| Test for Cholestasis | Sensitivity | Specificity | Source |
|---|---|---|---|
| Total Bilirubin | 98% | 93% | Dig Dis Sci. 1983 |
| | | | February; 28(2):129-36 |
| ALT | 58% | 90% | Dig Dis Sci. 1983 |
| | | | February; 28(2):129-36 |
| AST | 80% | 92% | Dig Dis Sci. 1983 |
| | | | February; 28(2):129-36 |
| ALK Phos | 92% | 83% | Dig Dis Sci. 1983 |
| | | | February; 28(2):129-36 |

FIG. 3 shows the composite likelihood for cholestasis for the patient at 7% using an iterative Bayesian analysis in accordance with embodiments of the invention. Again, this result is easily understood and takes all test information into account, including sensitivity and specificity information for the tests in relation to cholestasis. In contrast, Table 4 shows the results that would be obtained if single Bayesian calculations were performed for each test result assuming the same pre-test likelihood of choleostasis at 40%. The Table 4 results are disparate, ranging from a low of 1.4% to a high of 87% that the patient has cholestasis.

TABLE 4

| | | Initial | Post-Test |
|---|---|---|---|
| Test for Cholestasis | Result | Probability | Probability |
| Total Bilirubin | NEG | 40% | 1.4% |
| ALT | POS | 40% | 80% |
| AST | POS | 40% | 87% |
| ALK Phos | NEG | 40% | 6% |

Not only does the composite likelihood for cholestasis in FIG. 3 provide more meaningful information to a user, indicating the composite likelihood for two or more medical conditions—hepatocellular disease and choleostasis—it simultaneously allows the user to assess the relative likelihoods that a particular medical condition is present. While no definitive diagnosis is determined in FIG. 3, a clinician can use the composite likelihoods to evaluate whether any particular diagnosis is accurate, and base future testing around an appropriate hypothesis of alternate or additional disease states. For example, the results in FIG. 3 may be interpreted as indicating that the relative likelihood that the patient has hepatocellular disease is significantly higher than choleostasis. As a result, a clinician may focus additional tests and/or treatment more toward hepatocellular disease than choleostasis.

Embodiments of the invention may also be relatively insensitive to pre-test likelihood information, relaxing any need that a user provide highly accurate pre-test likelihood information for a Bayesian analysis conducted in accordance with inventive embodiments. As an example, suppose the user in the FIG. 3 embodiment did not assign a 51% initial likelihood for hepatocellular disease and 40% for cholestasis, but rather felt that each disease state was equally likely, assigning 30% pre-test likelihoods for each. In this case, the iterative Bayesian analysis employed herein determines post-test likelihoods of 56% and 5% for hepatocellular disease and cholestasis, respectively. The final post-test likelihoods are different than that shown in FIG. 3, but the differences between the post-test likelihoods for the two medical conditions remain relatively disparate. These results may be interpreted to strongly suggest that the clinician should suspect hepatocellular disease over cholestasis. Furthermore, the user may understand that the 56% likelihood of hepatocellular disease is far from definitive, and may have a basis for ordering more tests and investigating other possible medical conditions. As yet another example, assume that the user in FIG. 3 was initially somewhat sure of a cholestasis diagnosis over hepatocellular disease, and assigned pre-test likelihoods of 25% for hepatocellular disease and 75% for cholestasis. The iterative Bayesian analysis employed herein determines post-test likelihoods of 50% for hepatocellular disease and 27% for cholestasis. Again, while the final post-test likelihoods are different, the user may take pause in any conclusion that choleostasis is present, given that a pre-test likelihood of 75% was reduced to a 27% post-test likelihood after Bayesian analysis. Thus, the user is more likely to interpret the analysis results as suggesting hepatocellular disease should not be eliminated, and more evidence may be needed to conclude choleostasis as the likely disease state.

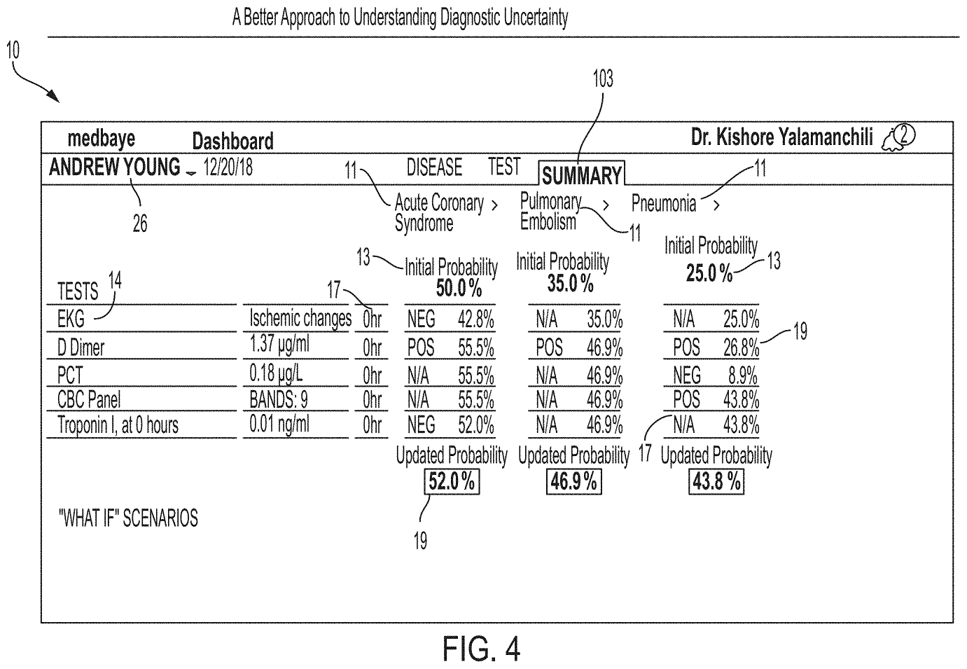

FIG. 4 shows another embodiment of a graphical user interface 10 that may be employed with inventive embodiments, including displaying post-test likelihoods for multiple medical conditions simultaneously and/or indicating post-test likelihoods for hypothetical tests and test results and/or determining post-test likelihoods for multiple medical conditions using a same set of test results. In this example, three medical conditions 11 are indicated, i.e., acute coronary syndrome (ACS), pulmonary embolism (PE) and Pneumonia. As with the embodiment of FIGS. 1-3, pre-test likelihoods 13 are indicated, along with multiple tests 14 and test results 17 for each medical condition 13. Post-test likelihoods 19 are indicated for each iteration of the Bayesian analysis of tests and test results, along with a composite likelihood for each medical condition 11. A patient 20 for whom the tests were performed and the analysis done is indicated as well. It should be appreciated that the graphical user interface 10 of FIG. 4 may be used in a medical record information system (e.g., an EMR system) that collects and stores information for multiple patients, e.g., as part of a hospital or other medical record information system including one or more databases. Thus, one of several different patients may be selected, e.g., using a drop-down menu, a search function, etc., and an analysis of medical conditions and tests and test results may be performed for any selected patient. Patients may be established in the record system in any suitable way, such as those employed to create a medical record for patients as a doctor's office, clinic and/or hospital. The medical record system may include information of various types, including patent name, address, and/or other identifying information, as well as tests and test results, past diagnoses and treatments, and so on.

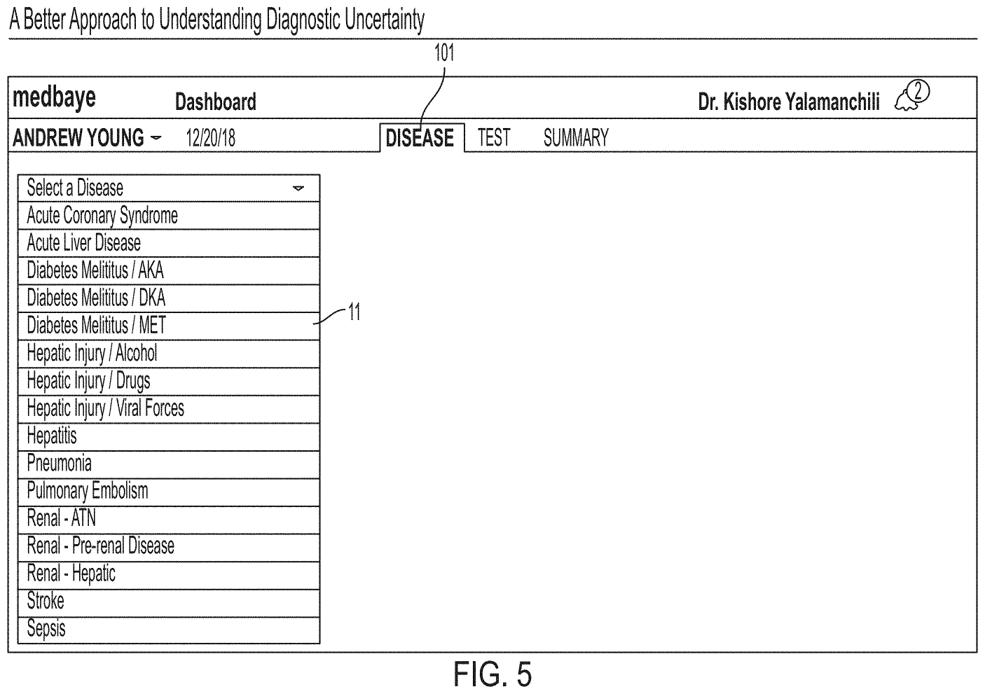

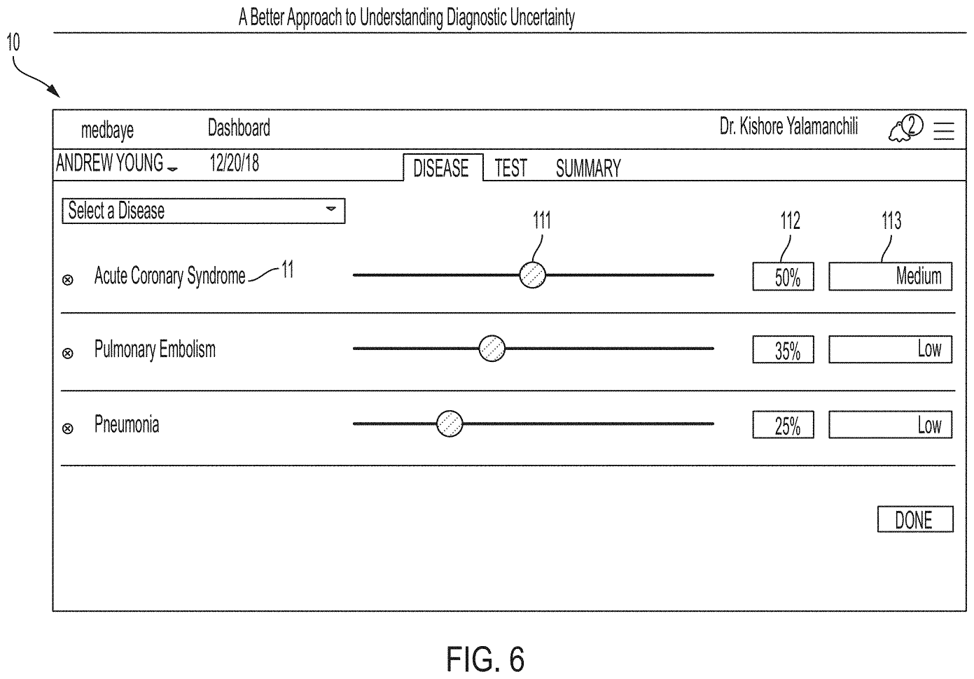

After a patient is selected or otherwise identified as being the subject of a medical condition likelihood assessment using the interface 10, one or more medical conditions may be identified for analysis. Initially, the “disease” tab 101 may be selected by the user or other action may be taken to indicate a desire to enter or otherwise identify one or more medical conditions 11 for assessment. Once on the “disease” tab 101 of the interface 10, one or more medical conditions 11 may be selected from a drop-down menu as shown in FIG. 5, and/or medical conditions may be identified in other ways such as by entering text into a suitable box of the interface 10 and/or automatically identified by the computerized system (e.g., based on tests that have been performed with respect to the patient). With one or more medical conditions 11 identified, a user may indicate a pre-test likelihood 13 for the medical condition, e.g., using a graphical user interface 10 as shown in FIG. 6. In this example, three identified medical conditions 11 are listed along with a corresponding slider 111, text box 112 and characterization box 113. A user may use any of the slider 111, text box 112 and characterization box 113 to indicate a pre-test likelihood 13 for the medical conditions 11, e.g., the slider 111 may be selected using a mouse or other pointing device (e.g., finger on a touch screen) and the slider 111 positioned to indicate the user's selection of the pre-test likelihood. A corresponding percentage value may be displayed in the text box 112 and/or characterization in the characterization box 113 in response to the user's positioning of the slider 111. Alternately, the boxes 112, 113 may remain blank. The user may alternately type a percentage value in the text box 112 and/or enter a characterization word (e.g., low, medium, high) into the characterization box 113 to indicate the pre-test likelihood rather than using the slider 111. In other arrangements, the computerized system may automatically provide a pre-test likelihood, such as based on epidemiological data, patient history, etc.

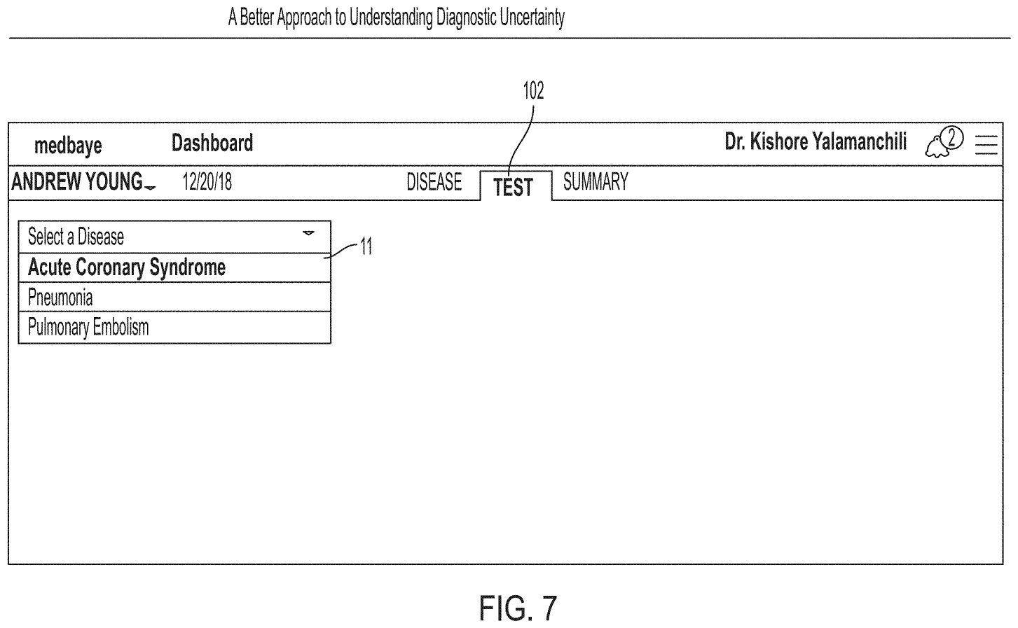

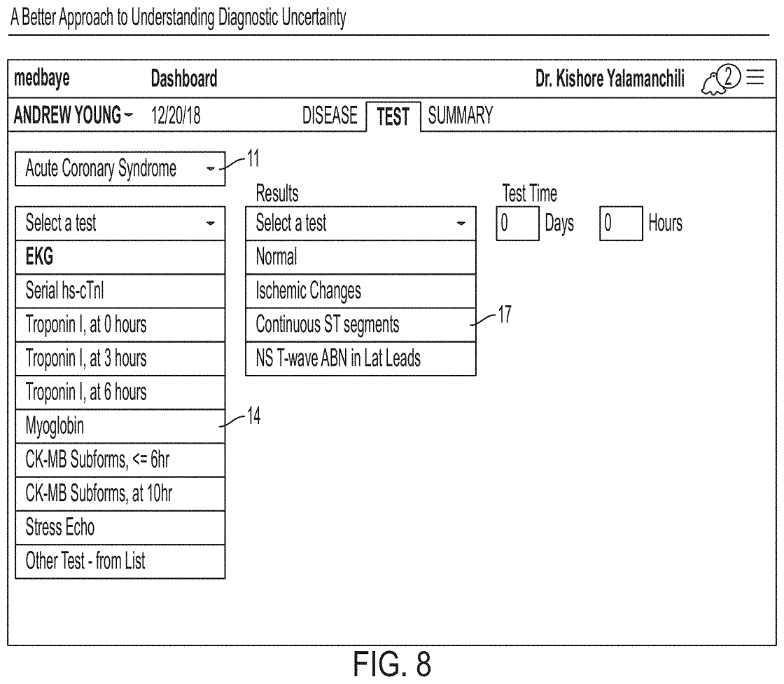

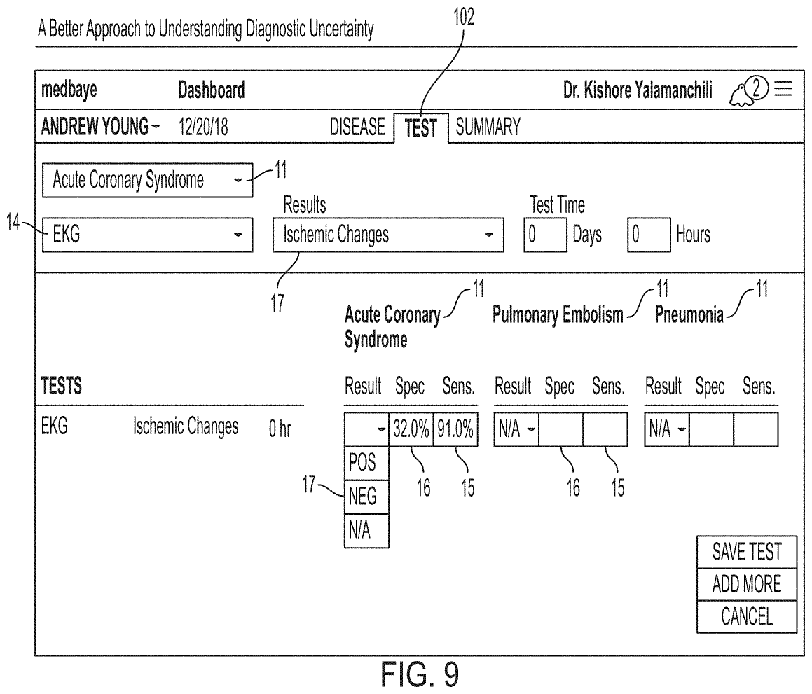

With one or more medical conditions and pre-test likelihoods identified, the user may next select the “test” tab 102 or otherwise indicate a desire to indicate one or more tests and test results for use in the analysis. On the test tab 102, one of the indicated medical conditions 11 may be selected as shown in FIG. 7, and in response, the interface 10 may permit the user to select or otherwise identify one or more tests 14, e.g., using a drop-down menu as in FIG. 8. One or more test results 17 may be indicated for each identified test, e.g., using a drop-down menu as shown in FIG. 8. Of course, tests and test results may be indicated in other ways, such as by automatically loading from a patient's medical record any and all tests and test results that may be relevant to a medical condition to be assessed. These automatically loaded tests and test results may be displayed on the interface 10, e.g., for modification and/or elimination by the user for purposes of the analysis. (Of course, any underlying medical record would not be modified or destroyed, only the test or result would be modified for purposes of medical condition analysis.) Other relevant information may be provided for the test and/or test result, such as a time that the test was performed, e.g., an absolute time, or elapsed time since a previous test, or elapsed time since the patient was first seen by a doctor, or other. As shown in FIG. 9, with a test identified, test result information 17 may be completed, in some cases for each corresponding medical condition 11. For example, particular tests may have different results for different medical conditions 11, and a user may indicate the different test results 17 for each medical condition 11. In some cases, the computerized system may normalize or convert test result data to a suitable test result 17 for purposes of medical condition analysis. As an example, a particular test result may be provided in terms of a number, e.g., a measurement in parts per million or percentage concentration, and the computerized system may convert the numerical test result to a “positive” or “negative” test result for purposes of analysis. Threshold values used to characterize numerical or other non-binary results as a binary result (e.g., negative or positive) may be defined by a user or other entity, e.g., values above the threshold may be characterized as “positive” and values below the threshold may be “negative.”

FIG. 9 illustrates that sensitivity and specificity information 15, 16 may be indicated for each test 14 and test result 17 for a corresponding medical condition 11. This information 15, 16 may be provided by a user, retrieved from a database of such information, or otherwise indicated. A user may be permitted to adjust sensitivity and specificity information 15, 16, if desired. A database from which sensitivity and specificity information 15, 16 is obtained may include sensitivity and specificity information 15, 16 for a wide variety of different tests and medical conditions, and may be determined based on clinical study or other data. Thus, the sensitivity and specificity information 15, 16 may represent state of the art data that is current and consistent with reliable clinical studies and other sources. By using such a database, users can be ensured that the most accurate and relevant sensitivity and specificity information 15, 16 is used for medical condition analysis. Moreover, the database may be maintained by a third party, e.g., separate from any entity with which the user is affiliated (such as a hospital), relieving the user and affiliated entity of having to verify sensitivity and specificity information 15, 16.

Returning to FIG. 4, with medical conditions and test information defined, a user may select the “summary” tab 103 of the interface 10 which indicates multiple tests 14 for multiple medical conditions 11, along with post-test likelihoods 19 determined by an iterative Bayesian analysis of the test results and sensitivity and specificity information 15, 16. As described above, the Bayesian analysis is performed in an iterative fashion, using the post-test likelihood of each iteration as the pre-test likelihood for the subsequent analysis. The final post-test likelihood determined is indicated as a composite likelihood of the medical condition. In FIG. 4, the composite likelihood for the ACS, PE and pneumonia is 52%, 46.9% and 43.8%, respectively. Note that some tests are not applicable to a particular medical condition, e.g., the PCT test is not applicable to ACS or PE, but is applicable to pneumonia. Where a test is not applicable to a medical condition, i.e., does not provide useful information to determine whether the medical condition is present or not, no change in the post-test likelihood is made as part of the Bayesian analysis. This can be done by suitably setting the test result and/or sensitivity and specificity for the test, e.g., at 50%/50%.

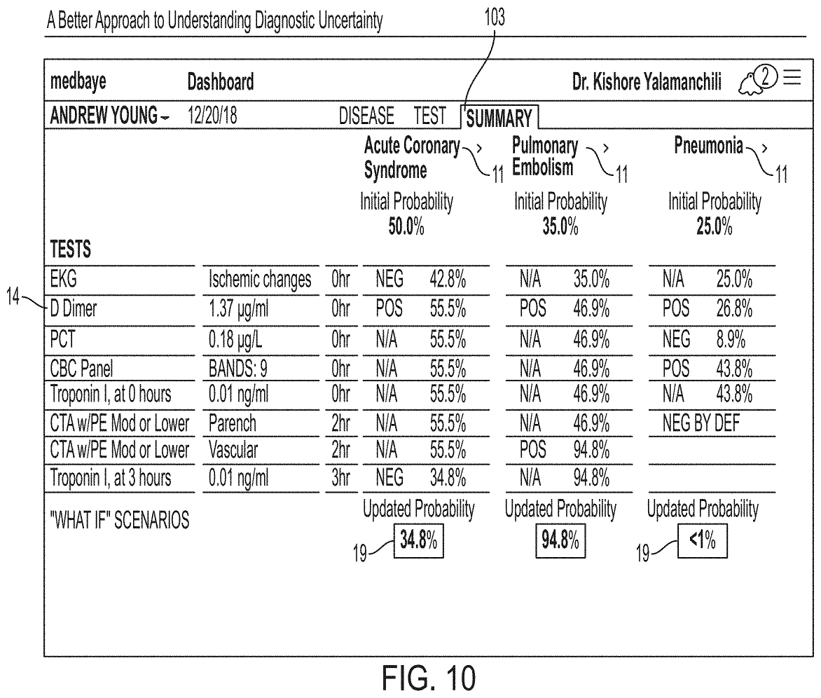

The post-test likelihood information in FIG. 4 could be interpreted by a user as indicating that all three medical conditions are approximately equal of being present, and so may prompt the user to perform additional tests, administer treatment, and/or assess alternate medical conditions. For example, as shown in FIG. 10, three additional tests 14 were performed. Their corresponding test results 17 and sensitivity/specificity information 15, 16 indicated, and post-test likelihoods 19 determined using the new test data. One of the tests 14 essentially ruled out the medical condition pneumonia, as the composite likelihood for pneumonia is indicated as being less than 1%. The additional tests also resulted in the composite likelihood for PE being increased to 94.8% and for ACS being reduced to 34.8%. These results may be interpreted as indicating that the patient more likely has PE than ACS.

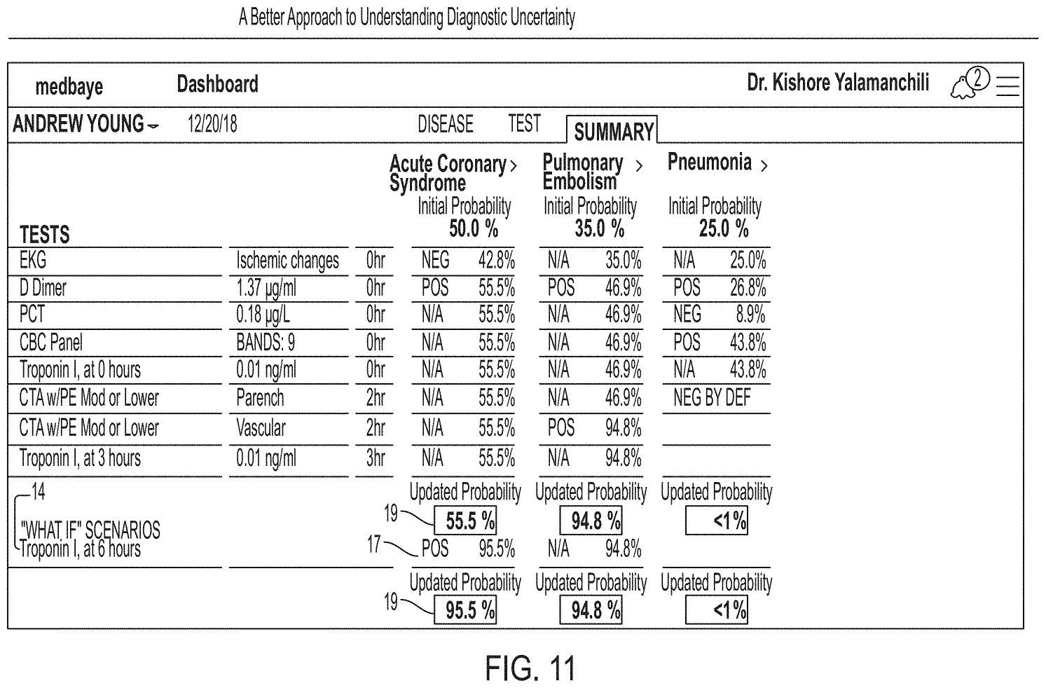

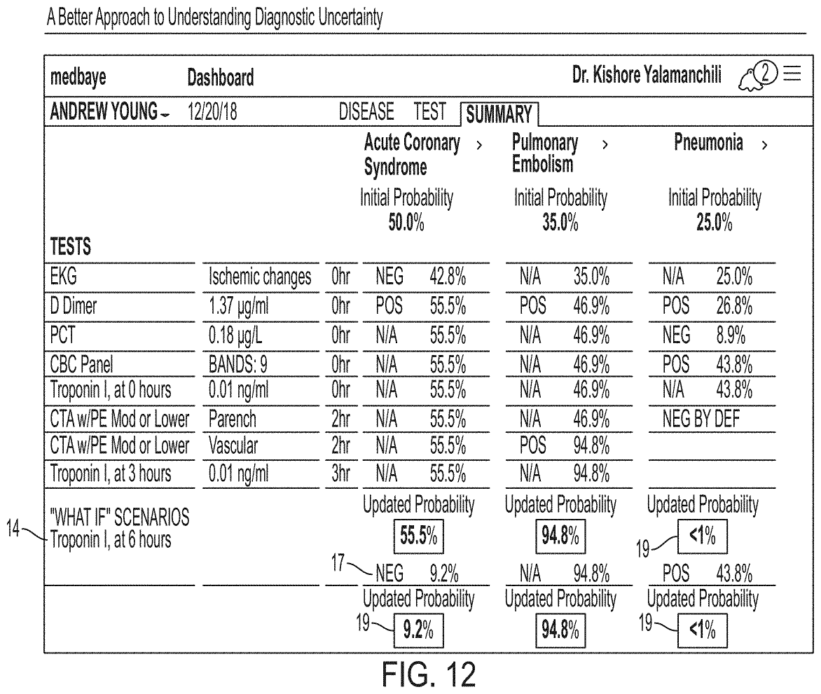

In some embodiments, a Bayesian analysis for a medical condition based on hypothetical tests and test results can be performed and post-test likelihood information for the hypothetical tests displayed. This can be extremely useful for a clinician when assessing whether to perform a test and/or understanding how a post-test likelihood for a medical condition will change based on different test results. This approach can also be useful in a teaching or training situation, even where all tests and test results are hypothetical. FIG. 11 shows an example where the user decided that the test results so far obtained in FIG. 10 did not provide a likelihood useful enough to exclude or otherwise assess ACS. Thus, the user decides to add the hypothetical results of a 6-hour Troponin I test 14, which has not yet been done, and evaluate the possible changes in likelihood for ACS that may result for both a positive 6-hour Troponin I test or a negative 6-hour Troponin I test. The results of a positive Troponin test (“POS”) are shown in FIG. 11, and the new post-test (composite) likelihood 19 determined for the hypothetical test and result is indicated as 95.5%. In such a case, the user may be comfortable in treating the patient for ACS. The results of a negative Troponin test (“NEG”) are shown in FIG. 12, where the updated composite likelihood 19 is indicated as 9.2%. In this case, the user may be comfortable excluding ACS, i.e., determining that the patient is very unlikely to have ACS. After considering the effect on composite likelihood of both test results for the Troponin 6-hour test, a clinician may conclude that Troponin test at 6 hours should be performed, e.g., because the test result will effectively determine whether the patient has the medical condition ACS. Note that in both FIG. 11 and FIG. 12 the earlier Troponin I results 17 at 0-hours and 3-hours have been automatically changed to “N/A” as compared to the test result used in FIG. 4. In this case the computerized system is configured such that serial Troponin tests are considered a single test determined by multiple results. The possible results are either “all negative”, recorded as a single negative test result, or “any one positive”, resulting in a single positive test result. Therefore, the prior Troponin tests before the hypothetical 6-hour test are listed as N/A to avoid double-counting the test results. A clinician may also make a change of this type to test results, e.g., where the clinician decides that the test for some reason may have been inaccurate, non-independent, or otherwise does not provide useful results. Alternately, the computerized system may automatically adjust a test result value as appropriate. For example, the computerized system may access stored information that indicates that this particular test does not provide useful information in analyzing a particular medical condition. In such a case, the test result may be adjusted to “N/A” or other value that has no effect on likelihood determination. In other embodiments, the computerized system may adjust the test result value to ‘N/A” or other where the test is repeated at a later time (whether actually or hypothetically), as is the case here. For example, consider hourly blood glucose draws. As blood glucose levels change over time, earlier results would become not applicable in light of later blood glucose measurements.

Although in some embodiments prior test results from a same test may be adjusted to an “N/A” or other value to have no effect on likelihood determination, this need not be done in all cases. Generally, in the art of disease diagnosis it is discouraged to repeat a test. The statistical reasoning behind this is that the clinician is assumed to be incapable of being neutral in the ordering of a repeated test. Often, it is the case that the clinician may disagree with a prior test result. If a test result came back negative and the clinician believes that the test result should have been positive, the clinician may be more likely to order the test again than if the test result agreed with the clinician's pre-conceived diagnosis. However, as long as the repeated test is independent to the original test (i.e. in the case of blood glucose, that a new blood draw is taken instead of the old sample being re-tested), duplicating a test may provide powerful statistical tool when evaluated dispassionately. The inventive system provides just such a format for considering duplicated tests. Consider a blood glucose test where the clinician is expecting to find a normal to elevated result, and instead a portable tester reads the glucose level of a finger stick as below normal. Normally, the clinician would order a blood draw to be read by laboratory equipment, as lab equipment is many times more sensitive and specific than a handheld unit. However, if we suppose that the portable tester has a sensitivity and specificity of 90%, and the lab equipment has a sensitivity and specificity of 98%, it is possible to avoid the more expensive and slower lab test by repeating the handheld test (with a new sample). As long as the two results agree (which they will around 90% of the time), the post-test likelihood of the two test results analyzed using the system in FIGS. 4-12 for a medical condition of “elevated glucose level” for example, will be more accurate than a single laboratory test. If we start at a 50% pre-test likelihood for the medical condition “elevated glucose level”, two negative tests from a handheld meter will lower the composite likelihood to 1.2%, whereas a single negative laboratory result will lower the probability to only 2.0%. It is often the case that repeating a lower cost test is often cheaper than a single higher quality test, and as long as the repeat tests are independent from one another (which may force other requirements, such as a wait time, etc.), accuracy may be increased over the standard protocols for differential diagnosis. Thus, the inventive method and/or system can provide an ability to use multiple tests, even tests of the same type, to assess the composite likelihood of a medical condition, e.g., in a way that avoids the use of higher cost tests.

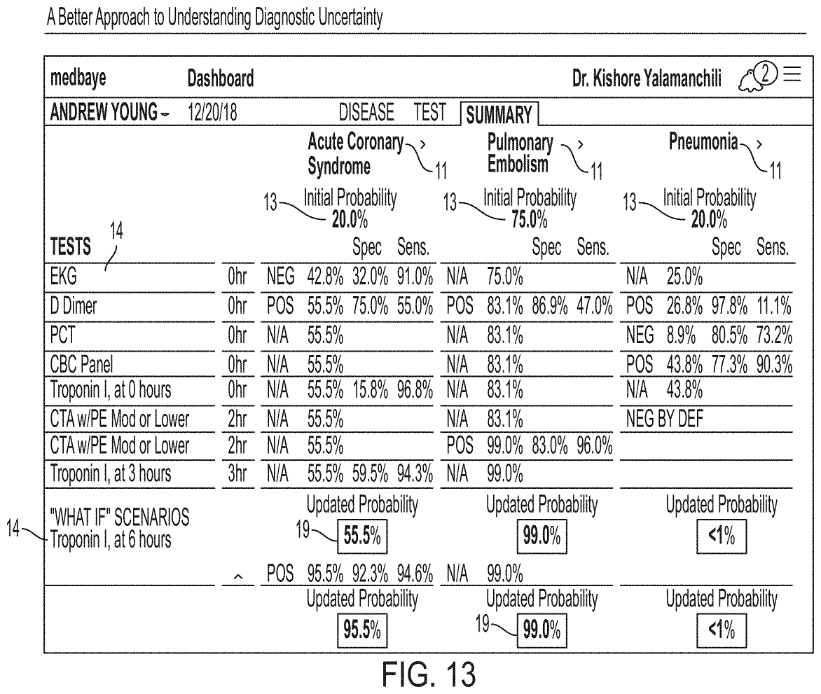

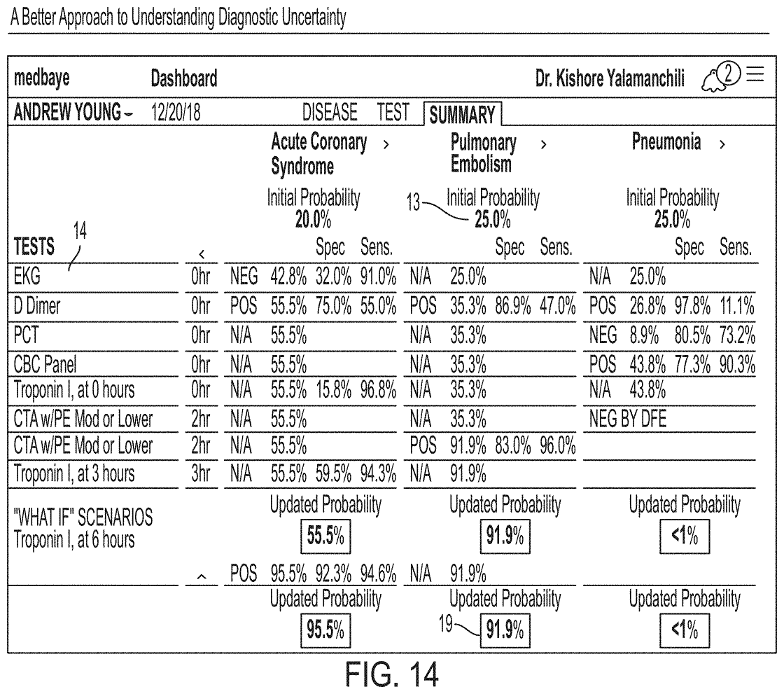

As noted above, the inventive medical condition assessment tool may allow a user to understand the extent, or lack thereof, to which a pre-test likelihood for a medical condition effects the composite likelihood for that medical condition. For example, a clinician may be concerned that an overly confident pre-test likelihood for a particular medical condition could skew the composite likelihood indicated by Bayesian analysis. The inventive method and system allows a user to make adjustments to a pre-test likelihood, even after multiple tests have been performed and assessed. For example, FIG. 13 shows the interface 10 of FIG. 11 where the user has adjusted the pre-test likelihood 13 for ACS to 20% (from 50%) and adjusted the pre-test likelihood 13 for PE to 75% (from 35%). As can be seen in FIG. 13, the composite likelihood 19 for ACS remains at 95.5%, and the composite likelihood 19 for PE increases to 99%. If the user wants to see the effect of lowering the pre-test likelihood for PE, she can do so as shown in FIG. 14 where the pre-test likelihood 13 for PE is dropped to 25%. The composite likelihood 19 for PE remains relatively high, at 91.9%, potentially giving confidence to the user that regardless of the pre-test likelihood 13 for PE, the iterative Bayesian analysis of the test results would still return a relatively high composite likelihood for PE.

The ability of the inventive method and/or system to receive information regarding hypothetical tests and test results can provide users with a powerful tool to assess whether tests will be useful or not in assessing whether a patient has a particular medical condition. The user may be permitted to assess multiple hypothetical tests and test results, as well as different combinations of such tests and test results, and understand the effect on the composite likelihood of multiple medical conditions, all without actually performing the tests. This ability may also permit a user to assess whether treatment should be administered or not, and whether tests done after treatment has at least been initiated will provide useful information regarding the patient's response to treatment. For example, a particular test may indicate that a potentially harmful compound is present in a patient, and a clinician may begin treatment to reduce the presence of the compound in the patient. While the clinician may know that the treatment will reduce the presence of the compound, the clinician may wonder whether the test should be repeated sometime after treatment begins so that the test results can be used to assess whether a particular medical condition is present or not. Using the hypothetical test and test results function of the inventive medical condition assessment tool, the clinician may be able to learn what effect different test results for the harmful compound will have on a likelihood for the medical condition. As another example, a clinician may diagnose a patient as having a particular medical condition and begin treatment for the condition. The clinician may use the hypothetical test and test result function to identify one or more tests which will be most effective in determining whether the patient is responding to the treatment. For example, the medical condition assessment tool may be used to identify tests which will indicate a reduction in composite likelihood for the diagnosed condition if the patient responds well to treatment, and will not indicate a reduction in composite likelihood if the patient does not respond well to treatment. Tests which will not provide useful information regarding response to treatment may be avoided.

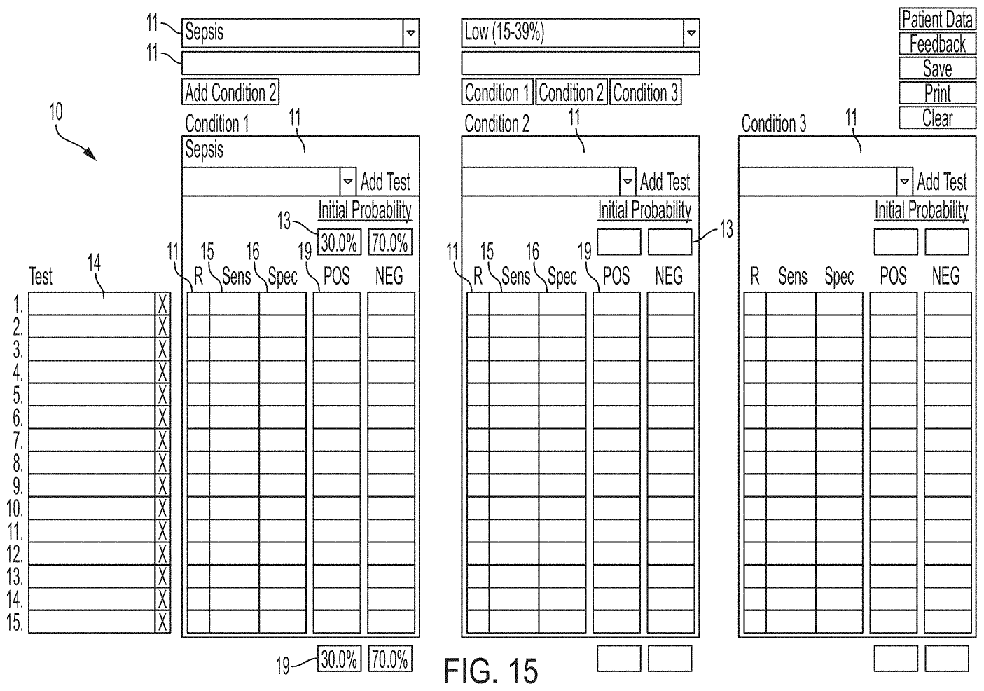

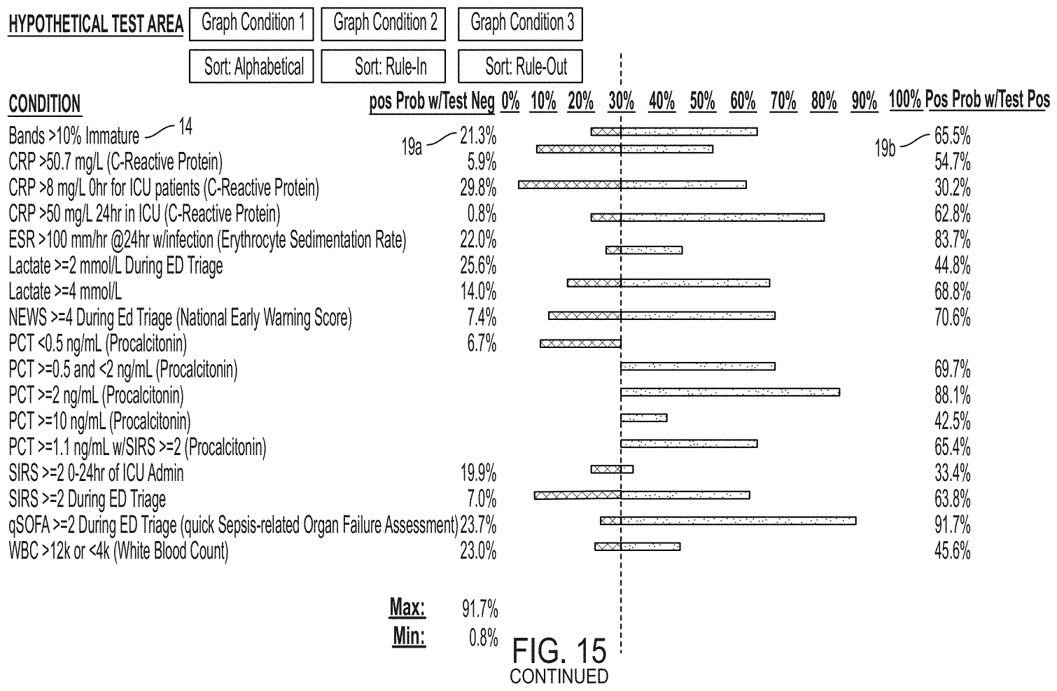

FIG. 15 shows a graphical user interface 10 display that includes elements of the FIG. 3 display. The upper part of the FIG. 15 display 10 includes one or more dialog boxes 11 to allow a user to select or otherwise define and display a medical condition (e.g., by typing the condition into a box 11, selecting the condition from a drop-down menu, etc.), and to define and display a pre-test probability 13. Multiple medical conditions may be simultaneously displayed along with corresponding pre-test probabilities 13 and other corresponding information, such as tests performed or could be performed 14, and test results 11 and sensitivity 15 and specificity 16 information for each test 14 and medical condition. The lower part of the FIG. 15 display includes one or more hypothetical tests 14 which are displayed. In this example, the hypothetical tests 14 are displayed in alphabetical order, but could be organized in other ways as discussed more below. Each hypothetical test 14 is displayed along with a corresponding composite likelihood for a negative test result 19a and a composite likelihood for a positive test results 19b. That is, the composite likelihood 19 that a corresponding medical condition is present is determined using the iterative Bayesian assessment for both a negative test result, i.e., composite likelihood 19a, and a positive test result, i.e., composite likelihood 19b. Thus, for each of the hypothetical tests 14 displayed, a user can readily see and understand the effect of a negative and/or positive test result. This example provides not only a numerical indication for the composite likelihoods 19a, 19b, but also a graphical representation which in this case includes a horizontal bar graph extending from the negative result composite likelihood 19a to the positive result composite likelihood 19b. The post-test composite likelihood 19 before the hypothetical test 14 is factored is also indicated by a vertical dashed line so the user can visualize the effect of a negative or positive test result on the composite likelihood 19a, 19b. This can be shown in other ways, such as by having the horizontal bar illustrated in one color when extending to the left of the dashed line, and in another color when extending to the right of the dashed line (in which case, the dashed line need not be illustrated at all). The use has the option to select one or more of the hypothetical tests 14, e.g., by double clicking on the test 14 in the display 10, and the selected test can be displayed in the upper portion of the display 10, i.e., in the numbered test boxes 14. This can allow a user to compile multiple hypothetical tests and determine a composite likelihood 19 for the compiled set of hypothetical tests. The use can also select whether to use a negative or positive test result (or other test result as appropriate and discussed above). The computerized system could automatically compile hypothetical test sets based on various criteria, e.g., four of the least expensive tests could be compiled and composite likelihood 19 determined for various test results, or a set of hypothetical tests that provide a highest or lowest composite likelihood 19 for a given medical condition could be determined and displayed for review by the user. In FIG. 15, the hypothetical tests 14 are displayed with composite likelihoods 19a, 19b for the single medical condition (“sepsis”) displayed, but the user has the option to display hypothetical tests 14 and composite likelihoods 19a, 19b for other medical conditions 11, e.g., by clicking a button on the display 10 to “graph condition 2” or “graph condition 3.”

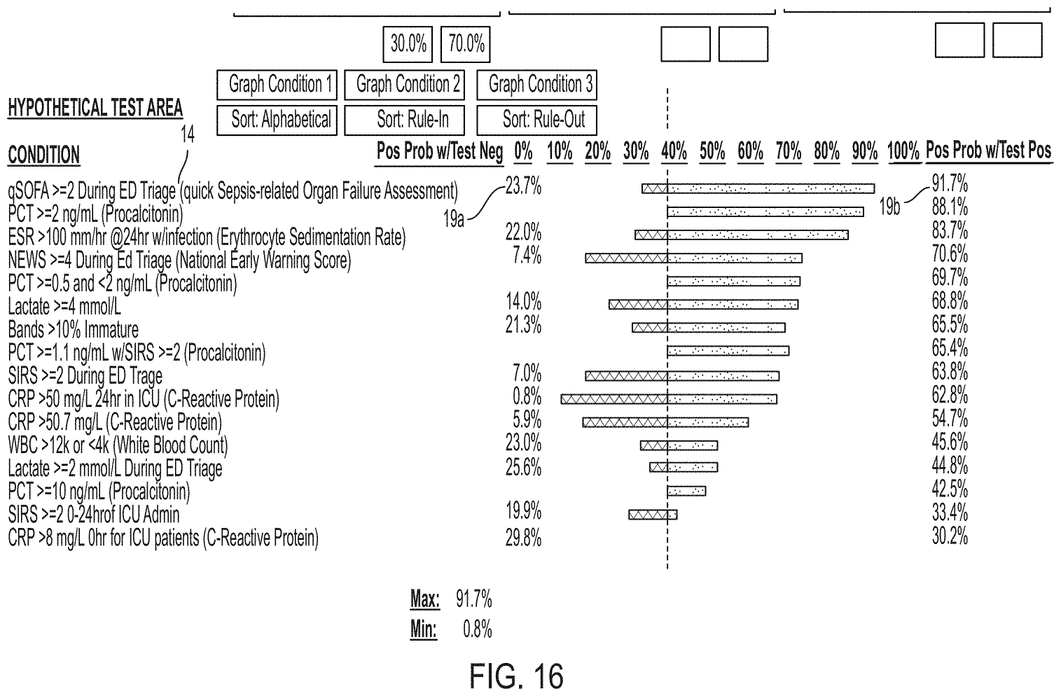

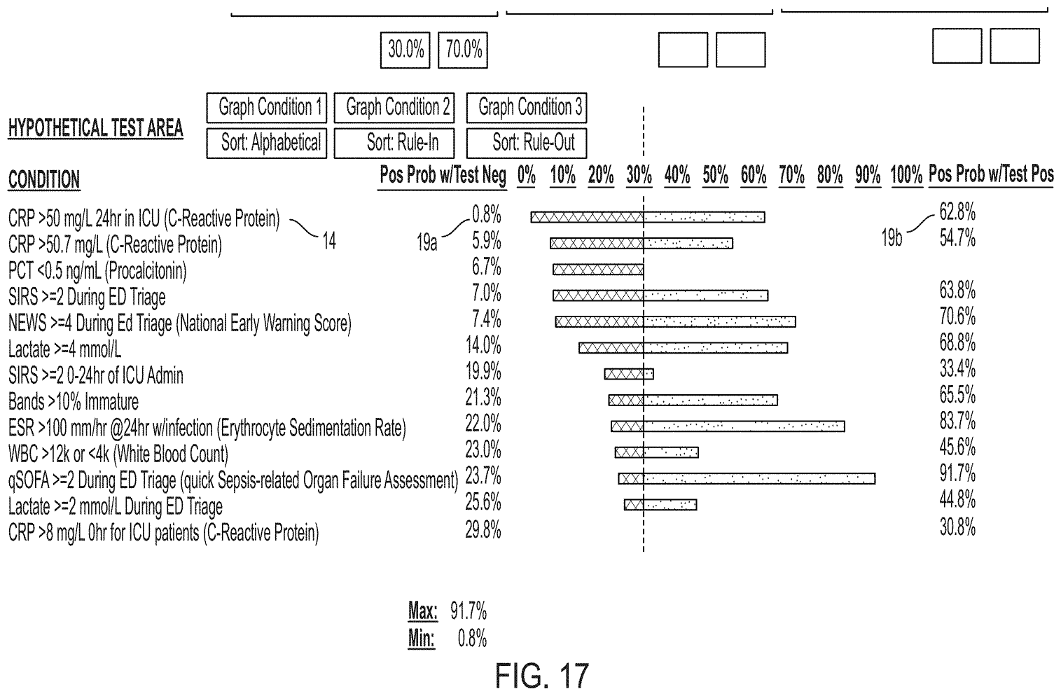

Hypothetical tests 14 may be displayed according to criteria other than alphabetical order as shown in FIG. 15. For example, FIG. 16 shows the lower portion of the graphical user interface of FIG. 15 with the hypothetical tests listed according to descending positive test result composite likelihood 19b. A user can cause this display by clicking a button “Sort: Rule-in” or other action, which causes the system to organize and display the hypothetical tests in descending positive test result composite likelihood 19b. This may help a user determine which test(s) may be most effective in “ruling-in” or identifying a medical condition as being relatively likely to be present. FIG. 17 shows the lower portion of the graphical user interface of FIG. 15 with the hypothetical tests listed according to ascending negative test result composite likelihood 19a. A user can cause this display by clicking a button “Sort: Rule-out” or other action, which causes the system to organize and display the hypothetical tests in ascending negative test result composite likelihood 19b. This may help a user determine which test(s) may be most effective in “ruling-out” or identifying a medical condition as being relatively unlikely to be present. Other displays are possible, such as sets of hypothetical tests that together provide a “rule-in” or “rule-out” assessment, hypothetical tests that provide lowest costs and most information regarding the presence or absence of a medical condition, etc. While FIG. 15 shows a display of hypothetical tests where no actual test information is included (e.g., where a patient first presents and a clinician is looking to identify tests that may be most useful for assessing one or more medical conditions), hypothetical tests and corresponding composite likelihoods 19a, 19b may be determined and displayed in situations where one or more tests have been conducted and results are included in the composite likelihood assessment, e.g., like that in FIG. 13.

In some embodiments, a computerized system may identify and display one or more tests that may be suggested for performance with respect to a patient, e.g., because the one or more tests are determined to provide useful information regarding whether a patient has a medical condition or not. Whereas in the example above where a user identifies a hypothetical test and test results for assessment, the computerized system may, using an iterative Bayesian analysis, determine composite likelihoods for multiple hypothetical tests and test results to identify one or more tests that should be performed or are at least suggests for performance, e.g., because of their potential value in diagnosing a patient. The computerized system may assess hundreds or thousands of possible tests or test combinations to identify those tests or combinations that can effectively rule-out, or rule-in, a particular medical condition, at least from the standpoint of a likelihood being above or below a particular threshold.

Similarly, the computerized system may identify and display one or more medical conditions as being relatively likely or unlikely to be present based on a set of tests and test results for a patient. Whereas in the example above a user identifies one or more medical conditions for assessment, the computerized system may instead, or in addition, assess post-test likelihoods using an iterative Bayesian analysis of the tests and test results to identify one or more medical conditions that have relatively high composite likelihoods, and therefore may be present in the patient. Such assessment may be performed for 10's, 100's, 1000's or more medical conditions using a same set of test results, including medical conditions that were never considered by a clinician. Consider, for example, a family of diseases that may have similar symptoms and may be differentially diagnosed. If the possible number of diseases that may be present in a patient is large, it may not be reasonable for the clinician to consider all diseases. Instead, only two or three diseases that a clinician feels are most likely or most important (more serious if the diagnosis is missed) may be considered by the clinician. If the clinician uses the application tool described herein to assess composite likelihoods of each disease based on test results for multiple tests, the application may additionally assess the composite likelihood for multiple other diseases that were not selected for inclusion by the clinician using the same test results. The application tool can use stored sensitivity and specificity information for each of the tests that corresponds to each of the additional diseases, as well as an estimated incidence rate as a pre-test likelihood for each disease to determine the composite likelihood for the additional diseases. If the application determines that the composite likelihood of one of these additional diseases reaches some threshold (perhaps defined based on the severity of each disease—and thus the danger is missing the diagnosis to the patient), or perhaps an arbitrary level (for instance if a probability is above a lowest probability of the disease(s) the clinician initially considered), then the application would alert the clinician of these results and allow the clinician to add or replace diseases as appropriate. This may give confirmation to a clinician that all relevant medical conditions have been considered (e.g., where the system fails to identify medical conditions having a higher likelihood than those already considered by the clinician), or identify alternatives to the clinician (e.g., of rare diagnoses that may be overlooked because of their infrequent occurrence). Medical conditions and corresponding test and test result information (including sensitivity, specificity and other information) used in this analysis may be stored in a database and retrieved by the computerized system for analysis.

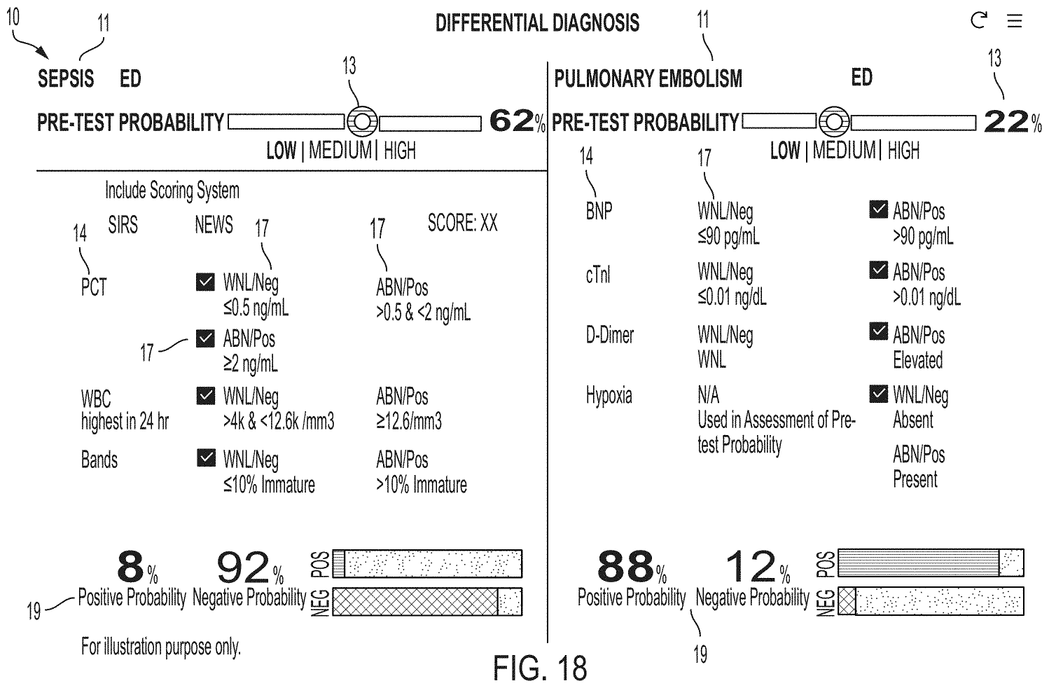

It will be appreciated that medical conditions, tests, test results, and other information may be presented to a user in a variety of different ways using a display of a graphical user interface. For example, FIG. 18 shows a display 10 that may be used by a clinician when assessing whether two (or more) medical conditions are present. In this example, two medical conditions 11 are displayed, along with corresponding pre-test likelihoods 13, which may be adjusted by a user by moving a slider element of the display 10. Multiple tests 14 are displayed for each medical condition 11, and the test results 17 may be actual test results, or may be hypothetical test results 17 and so may be selectable by a user. For example, a user may click or otherwise select a box for a desired test result 17, and a composite likelihood 19 will be determined based on the selected test result 17. As in other embodiments, additional tests 14 may be added or tests 14 may be removed from the assessment. Adjustment of any element of the display 10, such as adjustment of a test result 17 or pre-test likelihood 13, may cause a new composite likelihood 19 to be determined and displayed. Of course, the FIG. 18 display may be adjusted to display only one medical condition 11.

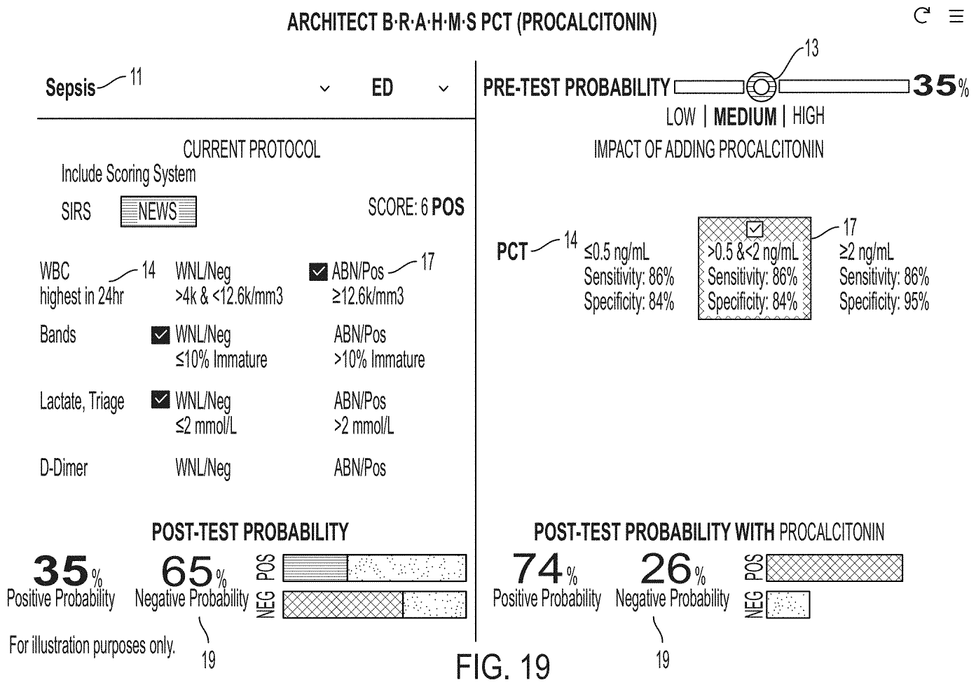

FIG. 19 shows another graphical user interface 10 including a medical condition 11, a set of hypothetical tests 14 and test results 17, and an additional hypothetical test 14 and test results 17. This display 10 is similar to those above, but illustrates how hypothetical tests and test results may be grouped in different sets to show the impact of performing one or more tests, or not. In this example, a set of hypothetical tests 14 and corresponding test results 17 are included on the left, and a single hypothetical test 14 and test result 17 (the “PCT” test) is on the right. Corresponding composite likelihoods 19 are determined and displayed below each set of tests. As in the example of FIG. 18, a user can select different test results 17 to see the impact of different test results on the resulting composite likelihood 19, as well as to compare the likelihoods 19 when the PCT test 14 is performed or not. Note in this example, the PCT test 14 can have any one of three possible results 17. A display 10 of this type may help a clinician assess the value of a particular test, including for different test result conditions.

As described above, tests actually or hypothetically performed with respect to a medical condition may assess whether a result of the medical condition is present (e.g., whether a particular compound is present, whether a particular set of symptoms are present, etc.) or whether a cause of the medical condition is present (e.g., whether a particular strain of bacteria is present). It is often the case that a patient having a certain medical condition meets the definition of having a second medical condition, but having the second condition does not ensure the patient has the first medical condition. For example, all patients in septic shock have sepsis, but not all sepsis patients are in septic shock Likewise, many medical conditions have multiple causes, which, regardless of the cause, have certain fixed results (e.g., abnormal blood panels, for instance). For many conditions, it is possible (or definitional) to diagnose a medical condition by only confirming the existence of a known cause with confirming symptoms, for instance bacteria in the bloodstream (e.g., bacteremia) with sepsis symptoms (or results) is the definition of sepsis, but having sepsis does not require bacteremia. Instead, the cause could alternatively be fungal or viral or other/unknown.

Diagnostic testing for complex medical conditions often focuses on both causes and results (e.g., symptoms or conditions) to diagnosis patients. For example, possible causes of sepsis in the blood may be bacterial, viral, fungal, or other/unknown. Tests focusing on the “results” of sepsis in the blood (regardless of the cause) might include procalcitonin, lactate, white blood count, and other. Using a Bayesian analysis on a diagnostic test for a “result”-type marker is straightforward, but the same analysis for a “cause” is not. Even a perfect test for bacteria in the blood stream that returns a negative result can only rule-out bacteremia. It cannot rule out sepsis as the patient might still have sepsis from another cause. There exist many usable published clinical studies that report a sensitivity and specificity of tests for bacteremia, but those numbers cannot be used if the clinician is only interested in understanding the likelihood that the patient has sepsis. If the test is positive, the assessment will indicate a likelihood that the patient has sepsis based on the sensitivity and specificity values for the test regarding the presence of bacteria in the blood, not whether the patient has sepsis. Even more problematic is a test result that is negative, i.e., which could exclude sepsis as being present whereas the test specificity and sensitivity relates to whether bacteria is present or not, not whether sepsis is present.

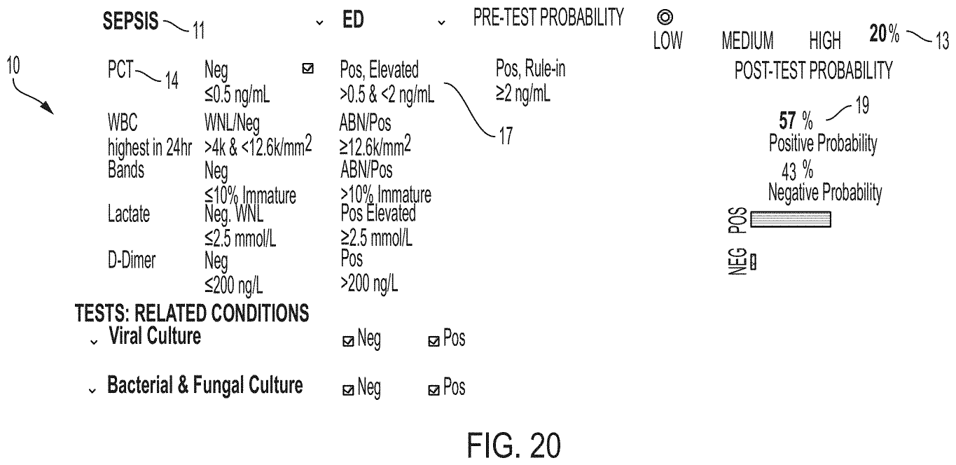

FIGS. 20-22 show example graphical user interfaces for a system that analyzes one or more tests for results of a medical condition as well as for causes and/or sub-conditions of the medical condition. In this example, tests for a medical condition's causes and results in a multi-test Bayesian analysis is performed even though the sensitivity and specificity for tests of the causes are for the presence of the cause and not for the medical condition of interest. To determine a composite likelihood for the medical condition based on the tests for causes of the medical condition having sensitivity and specificity information for the presence of the cause only, an estimated (or actual) fraction or percentage of cases of the medical condition in the patient population that come from the particular causes may be employed. (Causes of a medical condition are discussed below, but this assessment can be used for sub-conditions of a medical condition as well.) If the user is only aware of a fraction of causes for a medical condition that sum to less than 100%, the balance of 100% maybe be added as an “other/unknown” cause and the method may still be used to determine a composite likelihood for a medical condition. If a medical condition has causes that often are present together, such that the presence of each cause summed would be greater than 100%, the fractions may be reduced to the likely “primary” causes or if that is unknown to be equally reduced to sum to 100%. For instance, if a medical condition has three possible causes, and each cause is found in two-thirds of cases, the sum of three times two-thirds is six-thirds, or 200%. The fractions should therefore be reduced by half to one-third each, or another division that sums to 100% if additional data is available on prevalence.

To assess the value of this technique, consider that in a recent search of test sensitivity and specificity data for sepsis-related conditions, 42 high quality, large published studies of clinical trial results were found. An additional 76 high-quality studies were also located for tests that identify sepsis-related causes, such as bacteremia. However, these studies include sensitivity and specificity information for only the presence of the particular cause, not for whether sepsis is present or not. Being able to use these additional 76 tests for assessing the likelihood that sepsis is present provides the clinician with a much more powerful tool.

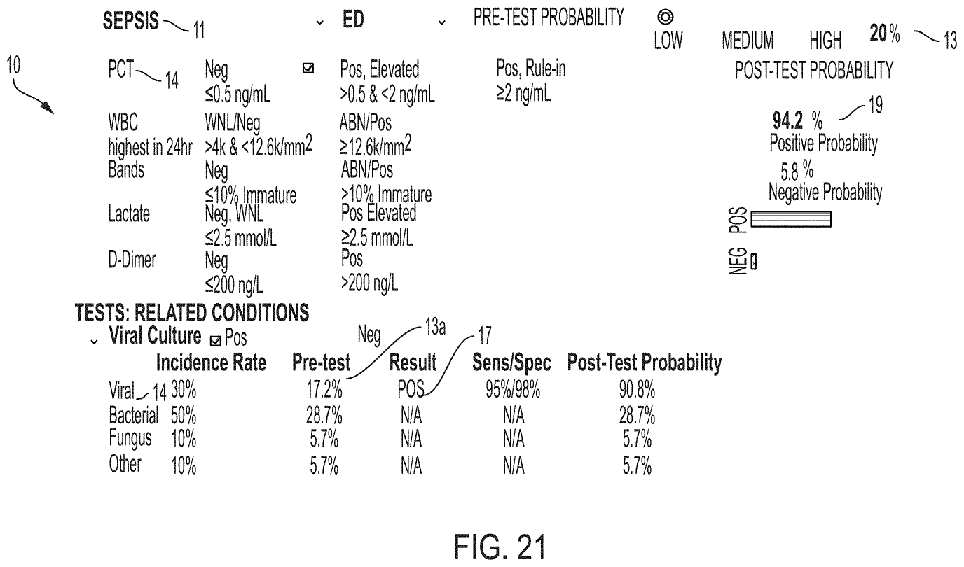

In FIGS. 20-22, a sepsis example is considered for a particular patient. In some cases of sepsis, the “cause” of the disease changes the way it is treated. For instance pneumonia is essentially sepsis of the lungs, and would be treated differently from sepsis of the blood. One test may culture bacteria from a sputum or other lung-derived sample, thereby testing whether a potential cause is located in the lungs. Another test may culture bacteria from a blood sample, and thus test whether a potential cause is located in the blood. Accordingly, determining the likelihood of a cause of a medical condition may provide value in addition to the value of assessing the likelihood that the medical condition is present. In this example, we assume all sepsis causes will be treated the same and so no particular focus is made regarding location of a potential medical condition cause, although the system may be adapted to do so. In this example of our patient population, the causes of sepsis of the blood is bacterial in 50% of cases, viral in 30% of cases, fungal in 10% of cases, and the remaining 10% is unknown/other. These are respective incidence values for each cause with respect to the medical condition of sepsis. Initially, the clinician (user) orders a test 14 (PCT) and enters the test result 17 into the user interface (0.5 and 2 ng/mL—a positive result) as well as the medical condition 11 (sepsis), and a pre-test likelihood 13 of sepsis at 20%, (e.g., based on the patient's symptoms and signs). Using the approaches described above, the user interface displays a composite likelihood 19 for sepsis (57%) based on the pre-test likelihood 13 and the test result 17 using sensitivity and specificity data (not shown) retrieved from a database for the PCT test 14 with respect to sepsis. The clinician is not satisfied with this level of uncertainty, and thus orders two other tests that assess the likelihood that one or more causes of sepsis are present. One of those tests is a viral culture, which is a test that does not have a known sensitivity or specificity for sepsis, but does have a good reference for “sepsis caused by viral forces” and a published sensitivity of 95% and specificity of 98% for the presence of a viral force. As shown in FIG. 21, the user enters the test result 17 for the viral culture test 14, which in this case is positive for a viral cause. The test results 17 for the viral culture test 14 are also entered for each of the other causes, (i.e., bacterial, etc.) but since the viral culture test 14 does not have relevant results for these causes, “N/A” is entered. Note, though that the viral culture test 14 is actually a test for each of the four causes in this case, and has respective test results for each cause. In this respect, the viral culture test 14 is effectively four different tests, one for each cause. The application retrieves from its database a group of relevant causes with their relative incidence fractions for sepsis for the relevant patient population. (Note that the user may enter this data or provide an estimate instead.) In this case, the application identifies that 30% of sepsis cases are caused by viral forces, 50% by bacteria in the bloodstream, 10% by fungal infections, and 10% by other causes as described above. Next the application determines the pre-test likelihood for sepsis for each of the four causes, identified by reference number 13a. As previously described, the overall pre-test likelihood for the four causes is the post-test probability from the PCT test of 57.3% from FIG. 20.

Since the patient is determined to have a 57.3% likelihood of having sepsis, the incidence fraction of each of the four causes is multiplied by 0.573 to find the four individual pre-test likelihoods 13a for each cause. This leads to a pre-test likelihood 13a of the viral culture of 17.2% for viral forces, 28.7% for bacteria, and 5.7% each for fungal and other sources (which sum to 57.3%). Now four different post-test likelihoods 19a are determined; one for each of the causes, and using the Bayesian techniques described above. The test for the viral culture was positive for viral forces, so a post-test likelihood 19a of 90.8% is determined from the 17.2% viral pre-test likelihood 13a and the 95% sensitivity and 98% specificity values 15, 16 for the test with respect to the presence of viral forces. The viral culture test 14 is not sensitive or specific for the other three causes, so a N/A, or not applicable, is automatically entered for each, and the post-test likelihood 19a for each cause is unchanged, i.e., are 28.7%, 5.7%, and 5.7% respectively. Note that these post-test likelihoods 19a represent the likelihood that each respective cause is responsible for the sepsis medical condition.

Next, a composite post-test likelihood 19 of sepsis is determined from the four post-test likelihoods 19a for each cause. The composite post-test likelihood 19 is determined as the union of the first through fourth post-test likelihoods 19a because each of these likelihoods 19a represents the likelihood that each cause is responsible for the medical condition. A typical union ∪ (defined as the probability of A or B or both) is:

P(A∪B)=P(A)+P(B)−P(A)P(B)

The union symbol U may also be written as OR. We are interested in the post-test likelihood of sepsis, which is the union of all causes of sepsis, in this case:

P(A∪B∪C∪D)=P(A)+P(B)+P(C)+P(D)−P(A∩B)−P(A∩C)−P(A∩D)−P(B∩C)−P(B∩D)−P(C∩D)+P(A∩B∩C)+P(A∩B∩D)+P(A∩C∩D)+P(B∩C∩D)−P(A∩B∩C∩D)

where ∩ represents an intersection. Setting A=90.8%, B=28.7%, C=D=5.7%, we get a post-test likelihood 19 of sepsis of 94.2%. Note that this is the composite likelihood of sepsis, not just sepsis from viral forces.

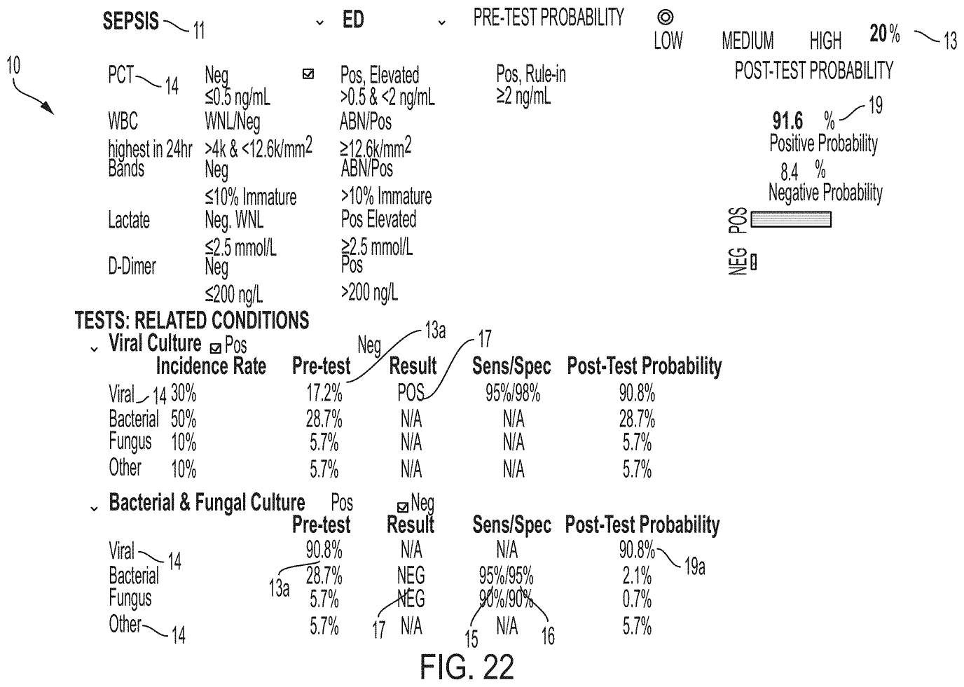

Assume next that the result for the second test for a potential cause of sepsis is returned. This test is a bacterial and fungal culture (bacteria and fungi are cultured together in this test, but the test has a different sensitivity and specificity for each cause). This bacterial and fungal culture returns a negative test result 17 for both bacteria and fungi, which is shown in FIG. 22. A test of this type might have different sensitivity and specificity values for different medical conditions and/or causes, e.g., one set of values for bacteremia, another for sepsis from fungal growth in the blood, and another for sepsis generally. Thus, a single test can be considered to be two or more tests for assessment purposes, e.g., one for each of multiple causes as well as one for an overall medical condition, and may have respective results for each cause and/or medical condition. The bacteremia sensitivity and specificity values 15, 16 are both 95% (as shown in FIG. 22), the fungal growth sensitivity and specificity 15, 16 are both 90% (as shown in FIG. 22), and the sepsis sensitivity is 40% and specificity is 98% (not shown in FIG. 22 but used to determine the composite likelihood 19 as discussed more below). Note that it is expected and common that the sensitivity for an overall main condition, such as sepsis in this case, will be relatively low as compared to sensitivity for one or more specific causes since many patients who get a negative test result from a test like this (e.g., for the presence of bacteria and fungus) may go on to have sepsis nonetheless. Although this is a fictitious example, it is typical that a test for a more specific cause (bacteremia) would be more accurate and more useful than the same test for a larger condition with various causes (sepsis) because the former will have clearer study inclusion and exclusion requirements, and require a small number of patients to be properly powered, and the study requirements to determine the overall sepsis sensitivity and specificity would be a larger and more expensive study to create an equivalent quality of data. The problem of course is that a clinician is not only interested in bacteremia, but rather in the overall medical condition of sepsis, and is not very concerned of the cause of sepsis, but only if it is present.

The post-test likelihood 19a is next determined for each of the four cause tests 14 resulting from the bacterial and fungal culture. The test for bacteria and fungus is not sensitive or specific to viral forces, and so the test result 14 is set to N/A and the post-test likelihood 19a for viral causes remains unchanged as 90.8%. The bacteremia pre-test likelihood 13a was 28.7%, and the negative result and sensitivity and specificity of 95% gives a post-test likelihood 19a for bacteremia-caused sepsis as 2.1%. The fungus pre-test likelihood 13a was 5.7%, and the negative result and sensitivity and specificity of 90% gives a post-test likelihood 19a of fungal-caused sepsis of 0.7%. Lastly the other causes of sepsis are also not applicable to this test and is its likelihood 19a remains at 5.7%.

Using the above approach for determining a composite likelihood 19 for sepsis, i.e., using a union of P(A∩B∩C∩D), gives a new overall post-test likelihood 19 for sepsis of 91.6%, down slightly from our 94.2% in FIG. 21 but still likely high enough to proceed with ordering therapy to treat sepsis.

Although not shown in FIG. 22, since the test for bacterial and fungus has sensitivity and specificity values for sepsis in general, a composite likelihood 19 for sepsis could be determined as discussed above using the negative test result for the bacterial and fungus test. In some embodiments, the application could determine the composite likelihood 19 as shown in in FIG. 22, and using the sensitivity and specificity values for sepsis in general and compare the results. For example, using the sensitivity and specificity values for sepsis in general, a composite likelihood 19 of sepsis of 90.9%, which is less than the composite likelihood 19 determined in FIG. 22. In some cases, both composite likelihoods may be displayed, and the user may have the option to switch between analysis methods. Alternatively, the application might be set to use which ever result was more definitive (i.e., closer to 100% if the probability is over 50%, or closer to 0% if the probability is under 50%). Using this criteria the user would see the 91.6% composite likelihood 19 over the 90.9% likelihood 19 as the 91.6% is closer to 100%. Another option would be to display results for the analysis that resulted in the biggest change in likelihood. This setting could be for a user who has clinical knowledge indicating a more appropriate patient population or test method makes that test more appropriate. As another option, the application might pick one method and test database entry over another or even exclude one based on which referenced study is best to use for this patient and patient population. The decision might be made based on the age of the study, size of the study, patient population or other characteristic. Lastly the user may have the option to see each reference and link directly to each study and decide which is the most appropriate analysis of the test results.

Note that the approach above regarding tests and test results for one or more causes of a medical condition may also be used for sub-conditions of a medical condition in addition or instead of causes. For example, just as a particular test may provide test results for a cause of a medical condition as well as for the presence of the medical condition (and may have different sensitivity and specificity values for each cause and/or the medical condition), a test may provide test results for one or more sub-conditions and/or a medical condition (and again have different sensitivity and specificity values for each sub-condition and/or the medical condition). Rather than being a cause of a medical condition, a sub-condition may be a symptom or other result that can at least some times be associated with a medical condition, such as a high body temperature may be a sub-condition of one or more medical conditions.

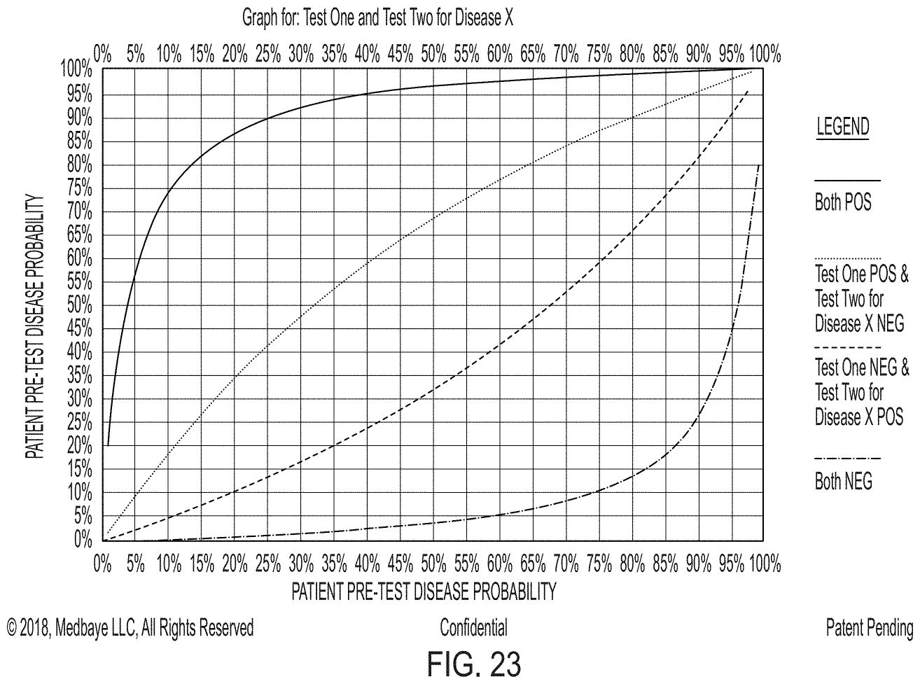

Another feature of the inventive methods and systems is the ability to display in graphical form a post-test likelihood for a medical condition for multiple tests and test results, as well as for multiple different pre-test likelihoods for the medical condition. FIG. 23 shows an illustrative example illustrating curves for the post-test likelihood for a medical condition regarding two tests. Four curves are shown, one each for possibilities of the test results being both negative, both positive, one negative and the other positive, and the one positive and the other negative. The curves are displayed for multiple different pre-test likelihoods for the medical condition, allowing a user to visualize how different test results, and combinations of test results effect the composite likelihood of the medical condition, as well as the effect of different pre-test likelihoods. Although an example for two tests is shown, any suitable number of tests may be included along with different combinations of test results.

There are several errors of human nature which a statistical analysis of disease probability may be helpful in revealing, teaching, and avoiding. Consider a patient considered for a disease with tests that disagree. Four tests have been given, with two returning positive, and two negatives. Assuming that the tests are of similar quality (which is often not the case), the post-test likelihood after the tests would be similar to the likelihood prior to the tests, meaning that we can assume the tests were ordered to alleviate some level of uncertainty over the diagnosis. However, in the confirmation bias that commonly exists in disease diagnosis, some clinicians will ignore or minimize the two tests that disagree with their preconceived notion, and over value the two tests the agree with the preconceived diagnosis, and interpret the composite results as supporting and concluding an unjustified minimization of the uncertainty.

Another common human error is assuming the over “count” of test results defines the posterior likelihood of diseases. Take the patient above but imagine that the four tests were split with three positive results and one negative result. As three is three times greater than one, even a dispassionate clinician might assume the patient has a very high probability of being positive—enough to initiate a treatment. But analyses as described herein can often disapprove that hypothesis.

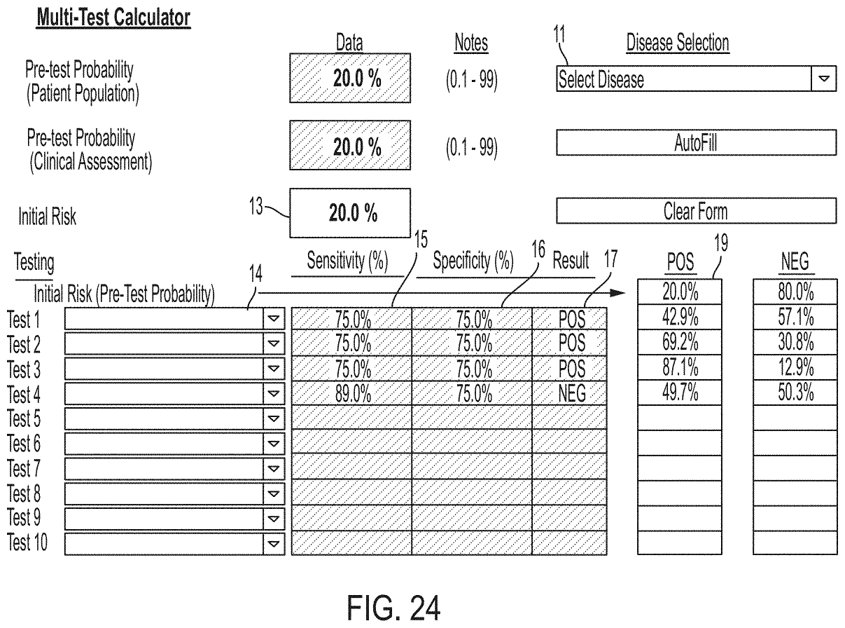

FIG. 24 illustrates the graphical user interface of FIG. 1 and helps convey the power of the iterative Bayesian analysis of tests and test results in inventive methods and systems. In this example, four tests have been completed, the first three with a positive result (POS) and having a sensitivity and specificity of 75% for the medical condition in question. A fourth test has been done with a negative (NEG) test result and sensitivity and specificity values of 89% and 75% for the medical condition. Initially, the patient was assessed with a 20% chance of being positive for the medical condition, i.e., the pre-test likelihood is 20%. With three solid and independent tests, each having a positive result, most clinicians would ignore the fourth result—perhaps calling it a flyer or an erroneous test result—and assume the likelihood that the patient has the medical condition as very high from the three positive test results.

However, the post-test likelihoods determined using the iterative Bayesian analysis according to embodiments of the invention are shown in FIG. 24 and highlight the error that would typically be made based on a rough assessment of the test results. From the initial 20% pre-test likelihood, the post-test likelihood rises to more than 40% once the first test alone is considered, and nearly 70% after the second, and 87% after the third test is considered. But the effect of the highly specific test four cannot and should not be ignored. All four tests, taken as a whole and employing the iterative Bayesian analysis herein, leave a less than 50% composite likelihood that the patient has the medical condition. A prudent clinician would stay open to alternate potential disease states and continue to monitor the patient. However, a prudent clinician without the composite likelihood generated in accordance with aspects of the invention would have little idea of the overall uncertainty in this common situation.

Input, output and other functions associated with the graphical interfaces 10 or other methods described herein may be implemented, at least in part, by a suitably programmed computer or other data processor, and may be employed in the form of software modules, ASICs, programmable arrays, or any other suitable arrangement, in addition to hardware components. For example, computer-implemented portions of a user device that employs the interface 10 may be implemented at least in part as single special purpose integrated circuits (e.g., ASICs), or an array of ASICs, each having a main or central processor section for overall, system-level control and separate sections dedicated to performing various different specific computations, functions and other processes under the control of the central processor section, as a plurality of separate dedicated programmable integrated or other electronic circuits or devices, e.g., hardwired electronic or logic circuits, such as discrete element circuits or programmable logic devices, as a programmed general purpose computer and/or other data processing device along with suitable software or other operating instructions, one or more memories (including non-transient storage media that may store software and/or other operating instructions), and so on. The devices may also include other components, such as an information display device, user input devices (such as a keyboard, user pointing device, touch screen, voice-activated control or other user interface), data storage devices, communication devices, a power supply for the control circuitry and/or other system components, temperature and other sensors (e.g., for detecting patient conditions), RFID and other machine-readable feature readers (such as those used to read and recognize alphanumeric text, barcodes, security inks, etc. which may be used to identify a patient wrist band, test reports, etc.), video recording devices, speakers or other sound emitting devices, input/output interfaces (e.g., such as the user interface to display information to a user and/or receive input from a user), communication buses or other links, a display, switches, relays, motors, mechanical linkages and/or actuators, or other components necessary to perform desired input/output or other functions.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method for providing information regarding a medical condition likelihood for a patient based on multiple test results, comprising:
receiving first information regarding a first test regarding the patient, the first information including a pre-test likelihood of the medical condition, a sensitivity for the first test with respect to the medical condition, a specificity for the first test with respect to the medical condition, and a first test result for the first test regarding the medical condition;
determining a first post-test likelihood of the medical condition based on the first information and using a Bayesian analysis of the first information;
receiving second information regarding a second test regarding the patient, the second information including a sensitivity for the second test with respect to the medical condition, a specificity for the second test with respect to the medical condition, and a second test result for the second test regarding the medical condition;
determining a second post-test likelihood of the medical condition based on the second information using a Bayesian analysis of the second information and using the determined first post-test likelihood for a second pre-test likelihood of the medical condition in the Bayesian analysis of the second information; and
displaying the second post-test likelihood as a composite likelihood that the patient has the medical condition based on the first and second tests.

2. The method of claim 1, wherein the first test is an actual test and the first test result is a test result that resulted from actually performing the first test with respect to the patient, and the second test is a hypothetical test that may be performed with respect to the patient and the second test result is a selected test result from multiple possible test results for the hypothetical test.

3. The method of claim 2, further comprising determining whether to actually perform the hypothetical test with respect to the patient based on the second post-test likelihood.

4. The method of claim 2, further comprising repeating the step of receiving second information regarding the second hypothetical test using a different selected test result for the second hypothetical test and determining and displaying a second post-test likelihood based on the different selected test result.

5. The method of claim 4, further comprising determining whether to actually perform the hypothetical test with respect to the patient based on the second post-test likelihood determined based on the different selected test result.

6. The method of claim 1, further comprising determining whether to perform a treatment on the patient for the medical condition based on the second post-test likelihood.

7. The method of claim 1, wherein the pre-test likelihood is a post-test likelihood determined using a Bayesian analysis of information for one or more tests regarding the patient.

8. The method of claim 1, further comprising determining that a treatment on the patient for the medical condition is unnecessary based on the second post-test likelihood.

9. The method of claim 1, further comprising determining that the patient has the medical condition based at least in part on the second post-test likelihood, and assessing an accuracy of the pre-test likelihood based on the determination that the patient has the medical condition.

10. The method of claim 1, wherein the medical condition is a first medical condition, the method further comprising:
receiving third information regarding the first test regarding the patient, the third information including a pre-test likelihood of a second medical condition that is different from the first medical condition, a sensitivity for the first test with respect to the second medical condition, a specificity for the first test with respect to the second medical condition, and a third test result for the first test regarding the second medical condition;
determining a first post-test likelihood of the second medical condition based on the third information and using a Bayesian analysis of the third information;
receiving fourth information regarding the second test regarding the patient, the fourth information including a sensitivity for the second test with respect to the second medical condition, a specificity for the second test with respect to the second medical condition, and a fourth test result for the second test regarding the second medical condition;
determining a second post-test likelihood of the second medical condition based on the fourth information using a Bayesian analysis of the fourth information and using the determined first post-test likelihood of the second medical condition for a second pre-test likelihood of the second medical condition in the Bayesian analysis of the fourth information; and
displaying the second post-test likelihood of the second medical condition as a composite likelihood that the patient has the second medical condition based on the first and second tests, the second post-test likelihood of the second medical condition being displayed simultaneously with the second post-test likelihood of the first medical condition.

11. The method of claim 10, wherein the first test is an actual test and the first and third test results are test results that resulted from actually performing the first test with respect to the patient, and the second test is a hypothetical test that may be performed with respect to the patient and the second and fourth test results are selected test results from multiple possible test results for the hypothetical test.

12. The method of claim 11, further comprising determining whether to actually perform the hypothetical test with respect to the patient based on the second post-test likelihoods of the first and second medical conditions.

13. The method of claim 11, further comprising repeating the steps of receiving second and fourth information regarding the second hypothetical test using different selected test results for the second hypothetical test and determining and displaying second post-test likelihoods of the first and second medical conditions based on the different selected test results.

14. The method of claim 11, further comprising determining that the patient has the first or second medical condition based at least in part on the second post-test likelihoods of the first and second medical conditions.

15. The method of claim 11, further comprising displaying the first, second, third and fourth test results and sensitives and specificities for the first and second tests for each of the first and second medical conditions simultaneously with the second post-test likelihoods of the first and second medical conditions.

16. The method of claim 11, wherein the pre-test likelihood of the first and second medical conditions is a post-test likelihood of the first and second medical conditions determined using a Bayesian analysis of information for one or more tests regarding the patient.

17. The method of claim 11, wherein one of the first and second tests does not test for the second medical condition, and one of the third and fourth test results has a corresponding value that results in no change in a post-test likelihood of the second medical condition resulting from the Bayesian analysis of the third or fourth information.

18. The method of claim 1, further comprising:
at least initiating a treatment for the medical condition on the patient at a time between when the first test is performed with respect to the patient and when the second test is performed with respect to the patient; and
comparing the first post-test likelihood with the second post-test likelihood to determine a response of the patient to the treatment.

19. The method of claim 18, wherein the second test is the same as the first test.

20. The method of claim 1, further comprising:
determining a plurality of third post-test likelihoods of the medical condition based on third information for a plurality of different tests using a Bayesian analysis of the third information and using the determined second post-test likelihood for a third pre-test likelihood of the medical condition in the Bayesian analysis of the third information, the third information for each of the plurality of different tests including a sensitivity for the test with respect to the medical condition, a specificity for the test with respect to the medical condition, and a hypothetical test result for the test regarding the medical condition; and
identifying and displaying one or more of the plurality of different tests as a recommended test to be performed with respect to the patient based on the third post-test likelihood for the one or more of the plurality of different tests.

21. The method of claim 20, wherein the step of identifying and displaying one or more of the plurality of different tests as a recommended test includes identifying tests of the plurality of different tests that have a corresponding third post-test likelihood that is either below a low threshold or above a high threshold.

22. The method of claim 1, further comprising:
determining composite post-test likelihoods for a plurality of possible medical conditions based on the first and second tests and corresponding sensitivity, specificity and test result information for the first and second tests with respect to each of the possible medical conditions, each of the composite post-test likelihoods for the plurality of possible medical conditions being determined by:
receiving information regarding the first test regarding the patient, the information including a pre-test likelihood of the possible medical condition, a sensitivity for the first test with respect to the possible medical condition, a specificity for the first test with respect to the possible medical condition, and a test result for the first test regarding the possible medical condition;
determining a post-test likelihood of the possible medical condition based on the information and using a Bayesian analysis of the information;
receiving information regarding the second test regarding the patient, the information including a sensitivity for the second test with respect to the possible medical condition, a specificity for the second test with respect to the possible medical condition, and a test result for the second test regarding the possible medical condition; and
determining a composite post-test likelihood of the medical condition based on the information regarding the second test using a Bayesian analysis of the information and using the determined post-test likelihood for a pre-test likelihood of the possible medical condition in the Bayesian analysis of the information regarding the second test.

23. The method of claim 22, further comprising:
displaying one or more of the possible medical conditions as a suggested medical condition for investigation based on the composite post-test likelihood for each of the one or more possible medical conditions; and
displaying the one or more possible medical conditions as a suggested medical condition simultaneously with the medical condition along with corresponding composite post-test likelihoods for the one or more possible medical conditions and the medical condition.

24. The method of claim 1, wherein the first test and the second test are hypothetical tests that may be performed with respect to the patient and the first test result and the second test result are selected test results from multiple possible test results for the hypothetical tests.

25. A method for providing information regarding treatment for a medical condition likelihood for a patient based on multiple test results, comprising:
receiving first information regarding a first test with respect to the patient, the first information including a first test result for the first test regarding the medical condition;
accessing a computer database for a sensitivity and a specificity for the first test regarding the medical condition, the computer database including sensitivity and specificity information for multiple tests regarding multiple medical conditions;
determining a first post-test likelihood of the medical condition using a Bayesian analysis of the first information and the sensitivity and specificity for the first test regarding the medical condition;
receiving second information regarding a second test with respect to the patient, the second information including a second test result for the second test regarding the medical condition;
accessing the computer database for a sensitivity and a specificity for the second test regarding the medical condition;
determining a second post-test likelihood of the medical condition using a Bayesian analysis of the second information and the sensitivity and specificity for the second test and using the determined first post-test likelihood for a second pre-test likelihood of the medical condition in the Bayesian analysis of the second information; and
displaying the second post-test likelihood as a composite likelihood that the patient has the medical condition based on the first and second tests.

26. The method of claim 25, wherein the first information includes a pre-test likelihood of the medical condition that is provided by a user.

27. The method of claim 25, wherein the first information includes a pre-test likelihood of the medical condition that is accessed from the computer database.

28. The method of claim 25, wherein the sensitivity and specificity information in the computer database is sourced from clinical trial information and randomized studies involving multiple subjects for multiple medical conditions.

29. The method of claim 25, wherein the medical condition is a first medical condition, the method further comprising:
receiving third information regarding the first test with respect to the patient, the third information including a third test result for the first test regarding a second medical condition that is different from the first medical condition;
accessing a computer database for a sensitivity and a specificity for the first test regarding the second medical condition;
determining a first post-test likelihood of the second medical condition using a Bayesian analysis of the third information and the sensitivity and specificity for the first test regarding the second medical condition;
receiving fourth information regarding the second test with respect to the patient, the fourth information including a fourth test result for the second test regarding the second medical condition;
accessing the computer database for a sensitivity and a specificity for the second test regarding the second medical condition;
determining a second post-test likelihood of the second medical condition using a Bayesian analysis of the fourth information and the sensitivity and specificity for the second test regarding the second medical condition and using the determined first post-test likelihood of the second medical condition for a second pre-test likelihood of the second medical condition in the Bayesian analysis of the fourth information; and
displaying the second post-test likelihood of the second medical condition as a composite likelihood that the patient has the second medical condition simultaneously with the second post-test likelihood of the first medical condition.

30. The method of claim 25, wherein the first test is an actual test and the first test result is a test result that resulted from actually performing the first test with respect to the patient, and the second test is a hypothetical test that may be performed with respect to the patient and the second test result is a selected test result from multiple possible test results for the hypothetical test.

31. A method for assessing likelihood of a medical condition that has multiple potential causes, comprising:
receiving first information regarding a first test regarding the patient, the first test for detecting whether a first cause of the medical condition is present, the first information including a first pre-test likelihood of the medical condition, a sensitivity for the first test with respect to the medical condition, a specificity for the first test with respect to the medical condition, and a first test result for the first test regarding the first cause;
determining a first post-test likelihood of the medical condition based on the first information and using a Bayesian analysis of the first information;
receiving second information regarding a second test regarding the patient, the second test for detecting whether a second cause of the medical condition is present, the second information including a second pre-test likelihood of the medical condition, a sensitivity for the second test with respect to the medical condition, a specificity for the second test with respect to the medical condition, and a second test result for the second test regarding the second cause;
determining a second post-test likelihood of the medical condition based on the second information and using a Bayesian analysis of the second information;
receiving third information regarding a third test regarding the patient, the third test for detecting whether a result of the medical condition is present, the third information including a sensitivity for the third test with respect to the medical condition, a specificity for the third test with respect to the medical condition, and a third test result for the third test regarding the medical condition;
determining a third post-test likelihood of the medical condition based on the third information using a Bayesian analysis of the third information and using a union of the determined first post-test likelihood and the determined second post-test likelihood for a third pre-test likelihood of the medical condition in the Bayesian analysis of the third information; and
displaying the third post-test likelihood as a composite likelihood that the patient has the medical condition based on the first, second and third tests.

32. The method of claim 31, wherein the sensitivity and specificity for the first test with respect to the medical condition is a sensitivity and specificity for the first test regarding whether the first cause is present, and the sensitivity and specificity for the second test with respect to the medical condition is a sensitivity and specificity for the second test regarding whether the second cause is present, wherein the first and second tests are a same test and the first and second test results are results regarding the presence of the first and second cause, respectively.

33. The method of claim 31, wherein the pre-test likelihood for the first and second pre-test likelihoods are each a respective likelihood that the first cause and the second cause are the cause of the medical condition, and wherein the first and second pre-test likelihoods are a fractional portion of a pre-test likelihood for the medical condition.

34. The method of claim 31, wherein the step of displaying the third post-test likelihood includes displaying the first and second post-test likelihoods simultaneously with the third post-test likelihood.

35-52. (canceled)
